SEQUENCE LISTING (30 sequences)

SEQ ID NO: 1 - protein, 10 aa, Artificial Sequence
  Synthetic amino acid sequence of heavy chain CDR1 of eculizumab (as defined under combined Kabat-Chothia definition)
GYIFSNYWIQ

SEQ ID NO: 2 - protein, 17 aa, Artificial Sequence
  Synthetic amino acid sequence of heavy chain CDR2 of eculizumab (as defined under Kabat definition)
EILPGSGSTEYTENFKD

SEQ ID NO: 3 - protein, 13 aa, Artificial Sequence
  Synthetic amino acid sequence of the heavy chain CDR3 of eculizumab (as defined under combined Kabat definition).
YFFGSSPNWYFDV

SEQ ID NO: 4 - protein, 11 aa, Artificial Sequence
  Synthetic amino acid sequence of the light chain CDR1 of eculizumab (as defined under Kabat definition)
GASENIYGALN

SEQ ID NO: 5 - protein, 7 aa, Artificial Sequence
  Synthetic amino acid sequence of light chain CDR2 of eculizumab (as defined under Kabat definition)
GATNLAD

SEQ ID NO: 6 - protein, 9 aa, Artificial Sequence
  Synthetic amino acid sequence of light chain CDR3 of eculizumab (as defined under Kabat definition)
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  Synthetic amino acid sequence of heavy chain constant region of eculizumab and BNJ421 antibody
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  Synthetic amino acid sequence of entire heavy chain of eculizumab
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  Synthetic amino acid sequence of heavy chain constant region of BNJ441 antibody
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  Synthetic amino acid sequence of entire heavy chain of BNJ441 antibody
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  Synthetic amino acid sequence of IgG2 heavy chain constant region variant comprising YTE substitutions
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  Synthetic amino acid sequence of entire heavy chain of eculizumab variant comprising heavy chain constant region depicted in SEQ ID NO15 (above)
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  Synthetic amino acid sequence of light chain CDR1 of eculizumab (as defined under Kabat definition) with glycine to histidine substitution at position 8 relative to SEQ ID NO4
GASENIYHALN

SEQ ID NO: 18 - protein, 17 aa, Artificial Sequence
  Synthetic depicts amino acid sequence of heavy chain CDR2 of eculizumab in which serine at position 8 relative to SEQ ID NO2 is substituted with histidine
EILPGSGHTEYTENFKD

SEQ ID NO: 19 - protein, 10 aa, Artificial Sequence
  Synthetic amino acid sequence of heavy chain CDR1 of eculizumab in which tyrosine at position 2 (relative to SEQ ID NO1) is substituted with histidine
GHIFSNYWIQ

SEQ ID NO: 20 - protein, 448 aa, Artificial Sequence
  Synthetic amino acid sequence of entire heavy chain of BNJ421 antibody
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 218 aa, Artificial Sequence
  Synthetic amino acid sequence of full light chain of BNJ383 antibody without signal peptide
DIQMTQSPSSLSASVGDRVTITCRASESVDSYGNSFMHWYQQKPGKAPKLLIYRASNLESGVPSRFSGSGSGTDFTLTISSLQPEDFATYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 112 aa, Artificial Sequence
  Synthetic amino acid sequence of light chain variable region of BNJ383 antibody
DIQMTQSPSSLSASVGDRVTITCRASESVDSYGNSFMHWYQQKPGKAPKLLIYRASNLESGVPSRFSGSGSGTDFTLTISSLQPEDFATYYCQQSNEDPYTFGGGTKVEIKR

SEQ ID NO: 23 - protein, 15 aa, Artificial Sequence
  Synthetic amino acid sequence of light chain variable region sequence Kabat LCDR1of BNJ383 antibody
RASESVDSYGNSFMH

SEQ ID NO: 24 - protein, 7 aa, Artificial Sequence
  Synthetic amino acid sequence of light chain variable region sequence Kabat LCDR2of BNJ383 antibody
RASNLES

SEQ ID NO: 25 - protein, 9 aa, Artificial Sequence
  Synthetic amino acid sequence of light chain variable region sequence Kabat LCDR3of BNJ383 antibody
QQSNEDPYT

SEQ ID NO: 26 - protein, 446 aa, Artificial Sequence
  Synthetic amino acid sequence of full heavy chain of BNJ383 antibody without signal peptide
QVQLVQSGAEVKKPGASVKVSCKASGYTFTDYSMDWVRQAPGQGLEWMGAIHLNTGYTNYNQKFKGRVTMTRDTSTSTVYMELSSLRSEDTAVYYCARGFYDGYSPMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 120 aa, Artificial Sequence
  Synthetic amino acid sequence of heavy chain variable region of BNJ383 antibody
QVQLVQSGAEVKKPGASVKVSCKASGYTFTDYSMDWVRQAPGQGLEWMGAIHLNTGYTNYNQKFKGRVTMTRDTSTSTVYMELSSLRSEDTAVYYCARGFYDGYSPMDYWGQGTTVTVSS

SEQ ID NO: 28 - protein, 5 aa, Artificial Sequence
  Synthetic amino acid sequence of heavy chain variable region sequence Kabat LCDR1of BNJ383 antibody
DYSMD

SEQ ID NO: 29 - protein, 17 aa, Artificial Sequence
  Synthetic amino acid sequence of heavy chain variable region sequence Kabat LCDR2of BNJ383 antibody
AIHLNTGYTNYNQKFKG

SEQ ID NO: 30 - protein, 11 aa, Artificial Sequence
  Synthetic amino acid sequence of heavy chain variable region sequence Kabat LCDR3of BNJ383 antibody
GFYDGYSPMDY

(19) United States
(12) Patent Application Publication
(10) Pub. No.: US 2020/0057046 A1
(43) Pub. Date: Feb. 20, 2020

(54) ASSAY FOR C5B-9 DEPOSITION IN COMPLEMENT-ASSOCIATED DISORDERS

(71) Applicant: ALEXION PHARMACEUTICALS, INC., Boston, MA (US)

(72) Inventors: Miriam GALBUSERA, Calusco D'adda (IT); Marina NORIS, Nembro (IT); Giuseppe REMUZZI, Bergamo (IT)

(73) Assignee: Alexion Pharmaceuticals, Inc., Boston, MA (US)

(21) Appl. No.: 16/342,462

(22) PCT Filed: Oct. 26, 2017
(86) PCT No.: PCT/US2017/058496

§ 371 Date: Apr. 16, 2019

Related U.S. Application Data
(60) Provisional application No. 62/413,614, filed on Oct. 27, 2016.

(51) Int. Cl. G01N 33/50; C07K 16/18

(52) U.S. Cl. CPC G01N 33/502; G01N 33/5091; C07K 2317/24; G01N 2800/52; G01N 2800/24; C07K 16/18

(57) ABSTRACT

Provided herein are methods of detecting C5b-9 deposition on endothelial cells. The methods are useful for screening patients for complement-associated disorders, for example, atypical hemolytic-uremic syndrome, as well as monitoring the efficacy of anti-C5 antibody therapy in a patient with a complement-associated disorder.

### CROSS-REFERENCE TO RELATED APPLICATIONS

This application claims priority to U.S. Provisional Application No. 62/413,614, filed Oct. 27, 2016. The contents of the aforementioned application are hereby incorporated by reference.

### BACKGROUND

Atypical hemolytic-uremic syndrome (aHUS) is a rare disease of unrestricted endothelial complement activation, which eventually causes renal microvascular thrombosis. It has an incidence rate of 1-2 new cases/million people/year.

Treatment of aHUS patients with the drug Soliris® is approved in the United States and in Europe. However, despite the availability of an effective drug for treating these patients, there remains a need to diagnose patients with aHUS, as well as to monitor the efficacy of treatment and course of the disease. The lack of specific and sensitive markers of complement activation not only applies to aHUS, but also to other complement-associated disorders.

A previous study described a test for the manual detection of C5b-9 deposition (Noris et al., Blood 2014; 124:1715-26). However, the test is cumbersome, time consuming, and can only be performed in advanced research laboratory settings by an expert Ph.D. scientist or a technician with many years of experience. Given that aHUS is a chronic disease and patients often require repeated tests to monitor the efficacy of eculizumab and other therapies and to monitor complement activity at the endothelial level throughout their lives, as well as to predict disease relapses if therapy is spaced or discontinued, there is an unmet need for reliable tests that are cost effective, reproducible, efficient, sensitive, accurate, and can be easily performed in non-advanced laboratory settings (e.g., by a technician with proper training). This would provide the important benefits of significantly reducing costs, and allowing patients to obtain their results more rapidly without the need for extensive travel. Such tests also would make outsourcing possible, and shorten the time of analysis, thereby rendering the test suitable for diagnosis, and treatment monitoring and adjustments. The methods described herein address these unmet needs.

### SUMMARY

Provided herein are methods for measuring complement C5b-9 deposition in patients with or suspected of having a complement-related disorder.

In one aspect, provided herein is a method for measuring complement C5b-9 deposition comprising:

(a) contacting ex vivo a biological sample obtained from a patient who has or is suspected of having a complement-associated disorder with disease-relevant cells;

(b) assessing levels of C5b-9 deposition on the cells;

(c) normalizing levels of C5b-9 deposition by cell number.

In another aspect, provided herein is a method for determining whether a patient with a complement-associated disorder (e.g., aHUS) would benefit from treatment with an inhibitor of C5 (e.g., eculizumab), the method comprising:

(a) incubating a biological sample obtained from the patient with and without an inhibitor of C5;

(b) contacting ex vivo endothelial cells with the biological sample from step (a);

(c) assessing levels of C5b-9 deposition on the cells; and

(d) normalizing levels of C5b-9 deposition by cell number, wherein less C5b-9 deposition with the biological sample incubated with the inhibitor compared to without the inhibitor indicates the patient is likely to benefit from treatment with the inhibitor.

In another aspect, provided herein is a method for monitoring a patient who has a complement-associated disorder and is being treated with an inhibitor of C5, the method comprising:

(a) contacting ex vivo endothelial cells with a biological sample from the patient and a control sample;

(b) assessing levels of C5b-9 deposition on the cells;

(c) normalizing levels of C5b-9 deposition by cell number; and

(d) increasing the dose of the inhibitor administered to the patient if C5b-9 deposition with the biological sample from the patient being treated with the inhibitor is greater compared to C5b-9 deposition with the control sample.

In one embodiment, if the patient is administered an increased dose of the inhibitor, steps (a)-(c) are repeated to determine whether the increased dose is sufficient to normalize levels of C5b-9 deposition on the cells.

In another aspect, provided herein is a method of treating a complement-associated disorder in a patient determined to be responsive to an inhibitor of C5 or eculizumab according to the methods described herein, the method of treatment comprising administering to the patient a therapeutically-effective amount of the inhibitor or eculizumab.

In some embodiments, the patient has atypical hemolytic uremic syndrome (aHUS), STEC-HUS, diabetes, lupus nephritis, vasculitis, or chronic allograft rejection.

In certain embodiments, the inhibitor of C5 is an antibody, such as eculizumab.

In some embodiments, the cells are cultured on a solid platform, such as a microplate (e.g., a 96-well microplate).

In some embodiments, the disease-relevant cells are selected from the group consisting of endothelial cells, retinal pigment epithelial cells, chondrocytes, neurons, glial cells, skeletal muscle cells, and cardiomyocytes. In some embodiments, the endothelial cells are selected from the group consisting of human microvascular endothelial cells from dermal origin, human umbilical vein endothelial cells, endothelial cells from foreskin, and endothelial cells from liver adenocarcinoma.

In certain embodiments, cells are plated at a density of about 5,000 to about 6,000 cells per well and cultured until confluent. In other embodiments, cells are plated at a density of about 10,000 cells to about 12,500 cells per well and cultured until confluent. In yet other embodiments, cells are plated at a density of about 15,000 cells per well cultured until confluent. In some embodiments, cells are confluent before being contacted with the biological sample (e.g., serum). In some embodiments, the serum is from a patient with aHUS, a patient in remission, or an eculizumab-naïve patient.

In some embodiments, cells are activated with adenosine 5′-diphosphate, thrombin, or lipopolysaccharide. In some embodiments, cells are contacted with the biological sample for about 1.5 hours to about 4 hours. In some embodiments, cells are incubated with a fixative such as paraformaldehyde after the contacting step but before the assessing step.

In certain embodiments, the levels of C5b-9 deposition in the methods described herein are assessed using an anti-C5b-9 antibody. In some embodiments, the anti-C5b-9 antibody is detected with a secondary antibody comprising a detectable label such as a dye. In some embodiments, the levels of C5b-9 deposition are assessed using an On-cell Western assay. In certain embodiments, cells are permeabilized after the anti-C5b-9 antibody is detected with the secondary antibody. In other embodiments, cells are permeabilized before the anti-C5b-9 antibody is detected with the secondary antibody. In some embodiments, following permeabilization, cells are incubated with an agent that accumulates in the nucleus, such as an agent that stains DNA. Exemplary agents include, for example, CellTag 700 Stain, DAPI, acridine orange, Hoechst 33342 Dye, Hoechst 33258, SYTOX Green nucleic acid stain, and Vybrant DyeCycle stain.

In certain embodiments, one or more steps of the methods described herein are automated.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows phase contrast microscopy images of 3,000, 4,000, 5,000, 6,000, 7,500, and 10,000 HMEC-1 cells cultured for different time points after seeding on 96-well microplates.

The last column on the right shows phase contrast microscopy images of cells stained with crystal violet dye after 96 hours of culture.

FIGS. 2A and 2B show phase contrast microscopy images of 10,000, 12,500, and 15,000 HMEC-1 cells cultured for different time points after seeding on 96-well microplates.

FIG. 3 shows a time course (24-96 hours) of HMEC-1 proliferation in 96-well plates.

FIGS. 4A-4D show representative images of viability experiments using HMEC-1 cells cultured for 96 hours.

FIG. 5A shows staining of resting HMEC-1 cells incubated with medium, control serum, aHUS1 acute serum, aHUS1 acute serum+sCR1, and aHUS2 acute serum. The secondary antibody was used at a 1:600 dilution. Left: 100 μL PBS/2% BSA blocking buffer; right: Odyssey blocking buffer.

FIG. 5B shows staining of ADP-activated HMEC-1 cells incubated with medium, control serum, aHUS1 acute serum, aHUS1 acute serum+sCR1, and aHUS2 acute serum. The secondary antibody was used at a 1:600 dilution. Left: 100 μL PBS/2% BSA blocking buffer; right: Odyssey blocking buffer.

FIG. 5C shows staining of resting HMEC-1 cells incubated with medium, control serum, aHUS1 acute serum, aHUS1 acute serum+sCR1, and aHUS2 acute serum. The secondary antibody was used at a 1:1200 dilution. Left: 100 μL PBS/2% BSA blocking buffer; right: Odyssey blocking buffer.

FIG. 5D shows staining of ADP-activated HMEC-1 cells incubated with medium, control serum, aHUS1 acute serum, aHUS1 acute serum+sCR1, and aHUS2 acute serum. The secondary antibody was used at 1:1200 dilution. Left: 100 μL PBS/2% BSA blocking buffer; right: Odyssey blocking buffer.

FIGS. 6A and 6B show staining with the CellTag 700 Stain (red) of resting HMEC-1 cells exposed for 4 hours to control or aHUS serum after 24 hour (left) or overnight (right) culture. A circle is drawn around the standard grid (FIG. 6A) and reduced grid (FIG. 6B).

FIG. 7 is a graph showing the correlation (R²=0.9696) between results of the classic C5b-9 test and automated C5b-9 test (overnight HMEC-1 culture, analysis with a standard grid).

FIG. 8 is a graph showing the correlation (R²=0.9631) between results of the classic C5b-9 test and automated C5b-9 test (24 hour HMEC-1 culture, analysis with a standard grid).

FIG. 9 is a graph showing the correlation (R²=0.73) between results of the classic C5b-9 test and automated C5b-9 test (overnight HMEC-1 culture, analysis with a standard grid).

### DETAILED DESCRIPTION

Disclosed herein are assays for detecting C5b-9 deposition on cells, e.g., endothelial cells, promoted by factors present in biological samples of patients with or suspected of having complement-associated disorders. The assays can be used, for example, to diagnose patients with complement-associate disorders, to determine whether patients are likely to benefit from treatment with an inhibitor of C5 (e.g., eculizumab), to titrate the dosage of an inhibitor of C5 in patients being treated with C5 inhibitors, and/or to screen for novel C5 inhibitors.

### Definitions

In order that the present description may be more readily understood, the following definitions are provided.

As used herein, the terms “polypeptide,” “peptide,” and “protein” are interchangable and mean any peptide-linked chain of amino acids, regardless of length or post-translational modification. The proteins described herein can contain or be wild-type proteins or can be variants that have not more than 50 (e.g., not more than one, two, three, four, five, six, seven, eight, nine, ten, 12, 15, 20, 25, 30, 35, 40, or 50) conservative amino acid substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

As used herein, the term “antibody” includes both whole antibodies and antigen-binding fragments of whole antibodies. Whole antibodies include different antibody isotypes including IgM, IgG, IgA, IgD, and IgE antibodies. The term “antibody” includes a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

As used herein, the term “antibody fragment,” “antigen-binding fragment,” or similar terms refer to a fragment of an antibody that retains the ability to bind to a target antigen (e.g., human C5) and inhibit the activity of the target antigen. Such fragments include, e.g., a single chain antibody, a single chain Fv fragment (scFv), an Fd fragment, an Fab fragment, an Fab′ fragment, or an F(ab′)₂ fragment. An scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al. (2001) J Immunol Methods 248(1):47-66; Hudson and Kortt (1999) J Immunol Methods 231(1):177-189; Poljak (1994) Structure 2(12):1121-1123; and Rondon and Marasco (1997) Annual Review of Microbiology 51:257-283, the disclosures of each of which are incorporated herein by reference in their entirety.

The term “antibody” includes, e.g., single domain antibodies such as camelized single domain antibodies. See, e.g., Muyldermans et al. (2001) Trends Biochem Sci 26:230-235; Nuttall et al. (2000) Curr Pharm Biotech 1:253-263; Riechmann et al. (1999) J Immunol Meth 231:25-38; PCT application publication nos. WO 94/04678 and WO 94/25591; and U.S. Pat. No. 6,005,079, all of which are incorporated herein by reference in their entireties. In some embodiments, the disclosure provides single domain antibodies comprising two VH domains with modifications such that single domain antibodies are formed. The term “antibody” also includes bispecific and multispecific antibodies which have binding specificities for at least two different antigens. Bispecific antibodies (including DVD-Ig antibodies) have binding specificities for at least two different antigens.

As used herein, the term “normal,” when used to modify the term “individual” or “subject” refers to an individual or group of individuals who does/do not have a particular disease or condition (e.g., aHUS) and is also not suspected of having or being at risk for developing the disease or condition.

As used herein, a “control sample” or “reference sample” refers to any clinical relevant control or reference sample, including, e.g., a sample from a healthy subject or a sample made at an earlier time from the subject being assessed. For example, a control sample or reference sample can be a sample from a subject prior to onset of a complement-associate disorder, at an earlier stage of disease, or prior to administration of treatment.

As used herein, “confluent” means that cells have formed a coherent monocellular layer on a surface (e.g., the surface of a well in a microplate), so that virtually all of the available surface is used. The term “substantially confluent” means that cells are in general contact on the surface, such that over about 70%, e.g., over about 90%, of the available surface is used. “Available surface” refers to a sufficient surface area to accommodate a cell.

As used herein, “complement-associated disorder” refers to all diseases and pathological conditions for which pathogenesis involves abnormal activation of the complement system.

As used herein, “eculizumab-naïve” refers to a patient who has not been previously treated with eculizumab.

As used herein, “biological sample” refers to fluids, cells, or tissues, and/or combinations thereof, isolated from a patient. In certain embodiments, the biological sample isolated from the patient is selected from the group consisting of serum, blood, and urine.

As used herein, “ex vivo” refers to an environment outside of a patient or subject.

The term “normalize,” as used in the context of “normalizing levels of C5b-9 deposition by cell number,” refers to obtaining raw C5b-9 signal levels in a cell sample (e.g., a well from a 96-well microplate) and dividing that value by the number of cells in the same cell sample. In the context of titrating the dose of an inhibitor of C5, the term “normalize” refers to increasing the dose of inhibitor until the level of C5b-9 deposition is essentially similar or lower to that of the control sample (i.e., baseline or background levels). In certain embodiments, a level of C5b-9 deposition essentially similar to that of the control sample may indicate a level of C5b-9 lower than that of the control, or, if higher, about 1-20% higher than that of the control, such as about 2%, about 3%, about 4%, about 5%, about 6%, about 7%, about 8%, about 9%, about 10%, about 11%, about 12%, about 13%, about 14%, about 15%, about 16%, about 17%, about 18%, about 19% or about 20% higher than that of the control.

As used herein, “patient” refers to a human or other mammalian subject who receives either prophylactic or therapeutic treatment.

As used herein, “subject” includes any human or non-human animal. “Non-human animal” refers to all vertebrates, e.g., mammals and non-mammals, such as non-human primates, sheep, dog, cow, chickens, amphibians, reptiles, etc.

The terms “therapeutically effective amount” or “therapeutically effective dose,” or similar terms used herein, are intended to mean an amount of an agent (e.g., an inhibitor of human C5) that will elicit the desired biological or medical response (e.g., an improvement in one or more symptoms of aHUS). In some embodiments, a composition described herein contains a therapeutically effective amount of an inhibitor of human complement component C5. In some embodiments, a composition described herein contains a therapeutically effective amount of an antibody, or antigen-binding fragment thereof, which binds to a complement component C5 protein. In some embodiments, the composition contains two or more (e.g., three, four, five, six, seven, eight, nine, 10, or 11 or more) different inhibitors of human complement component C5 such that the composition as a whole is therapeutically effective. For example, a composition can contain an antibody that binds to a human C5 protein and an siRNA that binds to, and promotes the degradation of, an mRNA encoding a human C5 protein, wherein the antibody and siRNA are each at a concentration that when combined are therapeutically effective. In some embodiments, the composition contains the inhibitor and one or more second active agents such that the composition as a whole is therapeutically effective. For example, the composition can contain an antibody that binds to a human C5 protein and another agent useful for treating or preventing a complement-associated disorder, such as aHUS.

As used herein “a”, “an”, and “the” include plural referents unless the context clearly indicates otherwise. The use of “or” or “and” means “and/or” unless stated otherwise. Furthermore use of the term “including” as well as other forms, such as “include,” “includes,” and “included,” is not limiting.

As used herein, “about,” when referring to a measurable value such as an amount, temporal duration, and the like, encompasses variations of up to ±10% from the specified value. Unless otherwise indicated, all numbers expressing quantities of ingredients, properties such as molecular weight, reaction conditions, scores in a scoring system, etc., used herein are understood as being modified by the term “about.”

Other features and advantages of the present disclosure will be apparent from the following description, the examples, and claims.

### I. Overview of Complement System

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum.

Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16^{th} Edition.

The complement cascade progresses via the classical pathway, the alternative pathway, or the lectin pathway. These pathways share many components, and while they differ in their initial steps, they converge and share the same “terminal complement” components (C5 through C9) responsible for the activation and destruction of target cells.

The classical pathway (CP) is typically initiated by antibody recognition of, and binding to, an antigenic site on a target cell. The alternative pathway (AP) can be antibody independent, and can be initiated by certain molecules on pathogen surfaces. Additionally, the lectin pathway is typically initiated with binding of mannose-binding lectin (MBL) to high mannose substrates. These pathways converge at the point where complement component C3 is cleaved by an active protease to yield C3a and C3b. Other pathways activating complement attack can act later in the sequence of events leading to various aspects of complement function. C3a is an anaphylatoxin. C3b binds to bacterial and other cells, as well as to certain viruses and immune complexes, and tags them for removal from the circulation. This opsonic function of C3b is generally considered to be the most important anti-infective action of the complement system. C3b also forms a complex with other components unique to each pathway to form classical or alternative C5 convertase, which cleaves complement component C5 (hereinafter referred to as “C5”) into C5a and C5b.

Cleavage of C5 releases biologically active species such as for example C5a, a potent anaphylatoxin and chemotactic factor, and C5b which through a series of protein interactions leads to the formation of the lytic terminal complement complex, C5b-9. C5a and C5b-9 also have pleiotropic cell activating properties, by amplifying the release of downstream inflammatory factors, such as hydrolytic enzymes, reactive oxygen species, arachidonic acid metabolites and various cytokines.

C5b combines with C6, C7, and C8 to form the C5b-8 complex at the surface of the target cell. Upon binding of several C9 molecules, the membrane attack complex (MAC, C5b-9, terminal complement complex—TCC) is formed. When sufficient numbers of MACs insert into target cell membranes the openings they create (MAC pores) mediate rapid osmotic lysis of the target cells. Lower, non-lytic concentrations of MACs can produce other effects. In particular, membrane insertion of small numbers of the C5b-9 complexes into endothelial cells and platelets can cause deleterious cell activation. In some cases activation may precede cell lysis.

As mentioned above, C3a and C5a are activated complement components. These can trigger mast cell degranulation, which releases histamine from basophils and mast cells, and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation. C5a receptors are found on the surfaces of bronchial and alveolar epithelial cells and bronchial smooth muscle cells. C5a receptors have also been found on eosinophils, mast cells, monocytes, neutrophils, and activated lymphocytes.

### II. Detection of C5b-9 Deposition Ex Vivo

Provided herein are new ex vivo assays for detecting the deposition of C5b-9 on cells (e.g., endothelial cells). Such assays are particularly useful for identifying patients with or suspected of having a complement-associated disorder who would be responsive to anti-C5 antibody therapy (e.g., by testing the ability of biological samples from such patients to promote C5b-9 deposition on cells). Such assays are also useful for screening candidate inhibitors of the alternate complement pathway, e.g., candidate inhibitors of C5.

In general, assays for detecting C5b-9 deposition on cells comprise the following steps:

(a) contacting ex vivo a biological sample obtained from a patient who has or is suspected of having a complement-associated disorder with a cell type relevant to the particular disorder of interest;

(b) assessing levels of C5b-9 deposition on the cells;

(c) normalizing levels of C5b-9 deposition by cell number.

The contacting step entails providing, ex vivo, cells relevant to the complement-associated disorder of interest, and contacting the cells with a biological sample from a patient who has or is suspected of having a complement-associated disorder. Without being bound by theory, complement molecules present in the biological sample promote the generation and deposition of C5b-9 on the cells. In a preferred embodiment, the disease of interest is aHUS, and a biological sample (e.g., serum) from the patient is contacted with endothelial cells. In one embodiment, the endothelial cells are HMEC-1 cells. In another embodiment, the endothelial cells are primary endothelial cells. In yet other embodiments, the endothelial cells are human endothelial cells of dermal origin, human umbilical vein endothelial cells, endothelial cells from foreskin, or endothelial cells from liver adenocarcinoma.

In one embodiment, podocytes or mesangial cells are contacted with a biological sample from a patient who has or is suspected of having a complement-associated disorder, such as C3 glomerulopathies. In another embodiment, retinal pigment epithelial cells are contacted with a biological sample from a patient who has or is suspected of having a complement-associated disorder, such as age-related macular degeneration. In another embodiment, chondrocytes are contacted with a biological sample from a patient who has or is suspected of having a complement-associated disorder, such as arthritis. In another embodiment, neurons and glial cells are contacted with a biological sample from a patient who has or is suspected of having a complement-associated disorder, such as multiple sclerosis, amyotrophic lateral sclerosis, Alzheimer's disease, and ischemic and traumatic brain injury. In another embodiment, erythrocytes are contacted with a biological sample from a patient who has or is suspected of having a complement-associated disorder, such as paroxysmal nocturnal hemoglobinuria. In another embodiment, skeletal muscle cells are contacted with a biological sample from a patient who has or is suspected of having a complement-associated disorder, such as myasthenia gravis. In another embodiment, cardiomyocytes are contacted with a biological sample from a patient who has or is suspected of having a complement-associated disorder, such as myocardial infarction. In a similar manner, any cell type known to be involved in a complement-associated disorder can be used in the methods described herein to test the ability of a biological sample from a patient with the disorder to promote C5b-9 deposition. The skilled artisan, using the guidance provided herein (in particular, in the Examples), could readily determine the conditions necessary to use a particular cell type in the methods described herein.

A biological sample from a patient can be, e.g., whole blood. In some embodiments, the biological fluid is a blood fraction, e.g., serum or plasma. In some embodiments, the biological fluid is urine. Additional suitable biological samples include samples comprising tissue, cell lysates, lymphatic fluid, saliva, cerebrospinal fluid, synovial fluid, nasal secretions, and other bodily fluids. Biological samples for use in the methods described herein are typically fresh, but can be stored frozen. Any biological sample that allows for/supports the generation of C5b-9 complex is suitable for use in the methods described herein. Whether a biological sample allows for/supports the generation of C5b-9 complex can be determined using methods known in the art and comparing with a positive control (e.g., a biological sample from a patient who is known to have a complement-associated disorder such as aHUS) and a negative control (a biological sample from a healthy control).

In some embodiments, the assay is performed on cells cultured on a solid support. The solid support can be, for example, beads, tubes, chips, resins, plates, wells, films, or microplates. Exemplary materials for the solid support include, but are not limited to, plastic, glass, ceramic, silicone, metal, cellulose, gels, polystyrene, polyester, and dextran. In a preferred embodiment, cells are cultured on a standard multiple-well microplate, such as a 96-well microplate.

In certain embodiments, the patient has or is suspected of having a complement-associated disorder selected from the group consisting of: rheumatoid arthritis (RA); antiphospholipid antibody syndrome (APS); lupus nephritis; ischemia-reperfusion injury; aHUS; typical (also referred to as diarrheal or infectious) hemolytic uremic syndrome associated with shiga-toxin-producing E. coli infection (STEC-HUS); dense deposit disease (DDD); neuromyelitis optica (NMO); multifocal motor neuropathy (MMN); multiple sclerosis (MS); macular degeneration (e.g., age-related macular degeneration); hemolysis, elevated liver enzymes, and low platelets (HELLP) syndrome; thrombotic thrombocytopenic purpura (TTP); spontaneous fetal loss; vasculitis (e.g., Pauci-immune vasculitis); glomerulopathies (e.g., C3 glomerulopathies); epidermolysis bullosa; chronic allograft rejection; recurrent fetal loss; traumatic brain injury; and injury resulting from myocardial infarction, cardiopulmonary bypass and hemodialysis. In some embodiments, the complement-associated disorder is a complement-associated vascular disorder such as a cardiovascular disorder, myocarditis, a cerebrovascular disorder, a peripheral (e.g., musculoskeletal) vascular disorder, a renovascular disorder, a mesenteric/enteric vascular disorder, vasculitis, Henoch-Schönlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease (arteritis), venous gas embolus (VGE), and restenosis following stent placement, rotational atherectomy, and percutaneous transluminal coronary angioplasty (PTCA). Additional complement-associated disorders include, without limitation, myasthenia gravis (MG), cold agglutinin disease (CAD), dermatomyositis, paroxysmal cold hemoglobinuria (PCH), Graves' disease, atherosclerosis, Alzheimer's disease, systemic inflammatory response sepsis, septic shock, spinal cord injury, glomerulonephritis, Hashimoto's thyroiditis, type I diabetes, psoriasis, pemphigus, autoimmune hemolytic anemia (AIHA), idiopathic thrombocytopenic purpura (ITP), Goodpasture syndrome, Degos disease, and catastrophic APS (CAPS). In a preferred embodiment, the patient has or is suspected of having aHUS or is in remission.

In some embodiments, the cells are contacted with the biological sample from the patient once they have formed a confluent monolayer on a solid support. For example, in one embodiment, endothelial cells are plated at a density of 5,000 cells/well on a 96-well microplate, and allowed to reach confluence prior to being contacted with the biological sample. Although dependent on cell culture conditions, HMEC-1 cells seeded at density of about 5,000 cells/well on a 96-well microplate typically take about 96 hours to reach confluence. In certain embodiments, endothelial cells are plated at a density of about 15,000 cells/well on a 96-well microplate. Again, although dependent on cell culture conditions, HMEC-1 cells seeded at density of about 15,000 cells/well on a 96-well microplate become confluent in about 16-24 hours. In certain embodiments, endothelial cells are plated at a density of about 10,000 or about 12,500 cells per well on a 96-well microplate. Although dependent on cell culture conditions, HMEC-1 cells seeded at about 10,000 or about 12,500 cells per well typically take about 48 hours to reach confluence. The duration from plating cells to reaching confluence depends on the cell type, and could readily be determined by the skilled artisan based on the guidance provided herein.

In further embodiments, cells are activated prior to being contacted with the biological sample. In some embodiments, cells (e.g., endothelial cells) are activated using adenosine diphosphate (ADP), lipopolysaccharide, or thrombin prior to being contacted with the biological sample. In other embodiments, cells are used in the resting state (i.e., cells are not activated).

In some embodiments, cells are seeded on a microplate in growth medium (i.e., medium containing serum), and cultured for a certain period (e.g., 24 hours) in medium without serum prior to being activated or contacted with the biological sample.

In some embodiments, for the contacting step, the biological sample (e.g., test serum) is mixed with medium (e.g., cell culture medium) at a volume/volume ratio of about 1:1 to about 1:10, for example, about 1:1 to about 1:8; about 1:1 to about 1:6; about 1:1 to about 1:4; or about 1:1 to about 1:2. In some embodiments, for the contacting step, the biological sample is mixed with medium at a volume/volume ratio of about 1:1, about 1:2, about 1:3, about 1:4, about 1:5; about 1:6, about 1:7, about 1:8, about 1:9, or about 1:10. In one embodiment, the biological sample is mixed with medium at a ratio of about 1:2. The mixture of biological sample and medium is herein referred to as the “test sample/test medium mixture.” In some embodiments, the test medium is Hank's buffered saline solution having a composition of, for example, 137 mmol/l NaCl, 5.4 mmol/l KCl, 0.7 mmol/l Na₂HPO₄, 0.73 mmol/l KH₂PO₄, 1.9 mmol/l CaCl₂, 0.8 mmol/l MgSO₄, 28 mmol/l Trizma base pH 7.3, 0.1% dextrose; with 0.5% BSA. Other suitable types of test medium include, for example, phosphate-buffered saline (PBS). Although not required, cells are typically washed with test medium (e.g., for one, two, three, or four times) prior to incubation with the test sample/test medium mixture.

In some embodiments, cells are contacted with the biological sample (e.g., test sample/test medium mixture) for about 30 minutes to 12 hours, for example, for about 1 hour to 8 hours, about 2 hours to 6 hours, or about 3 hours to 4 hours. In certain embodiments, the cells are contacted with the biological sample for about 30 minutes, about 1 hour, about 2 hours, about 3 hours, about 4 hours, about 5 hours, about 6 hours, about 7 hours, about 8 hours, about 9 hours, about 10 hours, about 11 hours, or about 12 hours. In a preferred embodiment, the cells are contacted with the biological sample for 4 hours.

Following the contacting step, cells may be fixed prior to the detection of C5b-9 deposition, particularly if they will be later subjected to immunostaining procedures. Suitable, non-limiting fixatives include, for example, paraformaldehyde, glutaraldehyde, formaldehyde, acetic acid, acetone, osmium tetroxide, chromic acid, mercuric chloride, picric acid, alcohols (e.g., methanol, ethanol), Gendre's fluid, Rossman's fluid, B5 fixative, Bouin's fluid, Carnoy's fixative, and methacarn. In a preferred embodiment, the cells are fixed in paraformaldehyde. In one embodiment, cells are fixed in 4% paraformaldehyde. Cells are typically washed following fixation with a suitable buffer, e.g., phosphate-buffered saline.

The assessing step typically involves the detection of C5b-9 deposition on cells (e.g., endothelial cells). In certain embodiments, the assessing step involves immunostaining (e.g., immunocytochemistry) with an antibody (i.e., a primary antibody) that specifically recognizes C5b-9 (e.g., an anti-C5b-9 antibody). Such antibodies are commercially available from, e.g., Calbiochem, or can be generated de novo using standard antibody production methods known in the art.

Prior to the detection of C5b-9 deposition, cells can be incubated with a blocking solution. Exemplary, non-limiting, blocking agents include bovine serum albumin, goat serum, fish skin gelatin, horse serum, swine serum, donkey serum, rabbit serum, or any suitable commercially-available blocking agent, such as Odyssey blocking buffer (LI-COR Biosciences).

The detection of C5b-9 deposition may involve immunostaining using primary and secondary antibodies. In some embodiments, the primary antibody is an antibody that specifically recognizes C5b-9 (e.g., human C5b-9). In one embodiment, the primary antibody is a polyclonal antibody. In another embodiment, the primary antibody is a monoclonal antibody. In some embodiments, the primary antibody can be from any species, e.g., rat, horse, goat, rabbit, mouse, guinea pig, human, etc.

In certain embodiments, the primary antibody is diluted in a suitable buffer at least at 1:50, for example, from about 1:50 to about 1:10,000, including 1:100, 1:150, 1:200, 1:250, 1:300, 1:350, 1:400, 1:450, 1:500, 1:550, 1:600, 1:650, 1:700, 1:750, 1:800, 1:850, 1:900, 1:1000, 1:1500, 1:2000, 1:2500, 1:3000, 1:3500, 1:4000, 1:4500, 1:5000, 1:6000, 1:7000, 1:8000, 1:9000, or about 1:10,000, and all ranges and values therebetween. Suitable buffers are well known to the skilled artisan, and include, for example, phosphate-buffered saline, Tris-buffered saline, and the like. In some embodiments, the buffer is supplemented with a detergent, for example, Triton X-100, NP-40, and the like, at a final concentration of about 0.05% to about 0.3%. for example, about 0.1%, about 0.15%, about 0.2%, about 0.25%, and all ranges and values therebetween, and/or a blocking agent (e.g., BSA).

The use of secondary antibodies to detect the binding between a primary antibody and an antigen is well-known (see, e.g., Antibodies: A Laboratory Manual, Harlow and Lane, Cold Spring Harbor laboratory Press, 1988; Current Protocols in Molecular Biology, Ausubel et al., John Wiley and Sons, Inc. NY, 2001). Secondary antibodies are chosen based on the species of origin of the primary antibody, e.g., if the primary antibody is a mouse antibody then the secondary antibody would be, for example, a rabbit anti-mouse antibody.

Secondary antibodies are typically coupled (e.g., conjugated or fused) to a detectable moiety. Detectable moieties can be conjugated to secondary antibodies using standard methods known in the art. Suitable detectable moieties include, but are not limited to, luminescent labels, fluorescent labels, radiolabels, enzymatic labels (e.g., horseradish peroxidase, alkaline phosphatase, beta-galactosidase, luciferase, urease, glucose oxidase, acetylcholinetransferase), chromophore labels, epitope tags, phosphorescent labels, ECL labels, dyes, haptens, bioten, photoaffinity labels, and the like. Secondary antibodies conjugated to detectable moieties for use in the methods described herein are also commercially available.

In some embodiments, the primary antibody is conjugated to a detectable moiety, and a secondary antibody is not used to detect C5b-9 deposits. Attachment of a detectable moiety does not interfere with the binding of the primary antibody to its target antigen (e.g., C5b-9). Methods for conjugating a detectable moiety to the primary antibody are routine in the art.

Following the binding of an antibody complex (primary/secondary antibody complex) to the antigen, C5b-9 deposition can be assessed by quantifying the signal generated from the antibody-antigen complex on cells. The means for detection is determined by the particular label used. For example, quantification may entail measuring a signal generated by a fluorescent dye conjugated to the primary or secondary antibody under a confocal microscope. Further, measuring may be performed after washing off, using a washing solution, antibodies which are not specifically bound to C5b-9. The washing solution may be, for example, selected from the group consisting of water, a buffer solution (e.g., PBS), a physiological saline, and a combination thereof. In certain embodiments, the measuring step is performed using an automated system, such as the On-Cell Western™ assay using the Odyssey CLX platform from LI-COR Biosciences. Details on how to perform and optimize the On-Cell Western™ assay are available at the manufacturer's website (www.licor.com). If using the On-Cell Western™ assay, in one embodiment, the secondary antibody is IRDye 800 CW goat anti-rabbit IgG (H+L) antibody (LI-COR), and is used at a dilution from about 1:500 to about 1:1500, for example, at about 1:600 or about 1:1200.

Once the level of C5b-9 deposition has been quantified with a means suitable for the detectable moiety used, the level of C5b-9 is normalized by the number of cells in the sample in order to eliminate variation due to differences in cell number. This can involve, for example, using a dye that binds to DNA (e.g., DAPI), the signal of which can be used to determine the number of cells in a sample. Other suitable agents for quantifying the number of cells in a sample include, e.g., acridine orange, Hoechst 33342 Dye, Hoechst 33258, SYTOX Green nucleic acid stain, and Vybrant DyeCycle Stain. Exemplary commercial stains include CellTag 700 stain from LI-COR and TO-PRO 3 (Life Technologies).

Cells, following fixation and prior to detection of C5b-9 deposition, may be subject to treatments that increase cell permeability to allow access of the agent used to determine the number of cells in a sample to intracellular compartments (e.g., the nucleus). Non-limiting agents which can be used to increase cell permeability include, for example, organic solvents, such as methanol and acetone, or detergents such as Triton-X 100, saponin, and Tween-20. In one embodiment, cells are permeabilized after the anti-C5b-9 antibody is detected with the secondary antibody conjugated to a detectable moiety. In another embodiment, if the anti-C5b-9 antibody itself comprises the detectable moiety, cells are permeabilized after detecting C5b-9 deposition with the anti-C5b-9 antibody. In yet another embodiment, cells are permeabilized before the anti-C5b-9 antibody is detected with the secondary antibody, or, if the anti-C5b-9 antibody itself comprises the detectable moiety, then before the anti-C5b-9 antibody is used to detect C5b-9 deposition on cells.

In some embodiment, one or more steps of the methods described herein (e.g., the measuring step described above) are automated, e.g., using an automated device to detect and quantify antibody staining patterns in a sample. In some embodiments, the plating of cells on a solid support is automated. In some embodiments, the contacting of cells with a biological sample is automated. In certain embodiments, immunostaining to detect C5b-9 deposits is automated (e.g., immunostaining). In some embodiments, measuring the levels of C5b-9 deposition is automated (e.g., using the On-cell Western™ assay with the Odyssey CLX platform from LI-COR, or any other platform that allows for automated detection/quantification of a detectable moiety, such as a fluorescent dye(s)), e.g., EnSight Multimode Plate Reader (PerkinElmer); Cytell Imaging System (GE Healthcare). In some steps, normalizing the levels of C5b-9 deposition by the number of cells is automated. In some embodiments, all steps are automated.

The methods described herein are typically performed in conjunction with a reference or control sample. In some embodiments, the control or reference sample is a corresponding biological sample from a healthy individual. In other embodiments, the control or reference sample is a biological sample obtained before a patient developed a complement-associated disorder. These control or reference samples can provide a standardized reference for the amount of C5b-9 deposition promoted by a biological sample. The methods described herein can also be performed in conjunction with a positive control, e.g., a biological sample from a patient known to have a complement-associated disorder.

### III. Methods of Diagnosis, Monitoring, and Treatment

Provided herein are methods for determining whether a patient with a complement-associated disorder would benefit from treatment with an inhibitor of C5, the method comprising:

(a) incubating a biological sample obtained from the patient with and without an inhibitor of C5;

(b) contacting ex vivo endothelial cells with the biological sample from step (a);

(c) assessing levels of C5b-9 deposition on the cells; and

(d) normalizing levels of C5b-9 deposition by cell number,

wherein less C5b-9 deposition with the biological sample incubated with the inhibitor compared to without the inhibitor indicates the patient is likely to benefit from treatment with the inhibitor.

In certain embodiments, the complement-associated disorder and cell type are any of those listed in the preceding section. In a preferred embodiment, the complement-associated disorder is aHUS. In one embodiment, the endothelial cells are HMEC-1 cells.

In some embodiments, the inhibitor of C5 is eculizumab. According, also provided herein are methods of determining whether a patient with a complement-associated disorder is likely to benefit from treatment with eculizumab, the method comprising:

(a) incubating a biological sample obtained from the patient with and without eculizumab;

(b) contacting ex vivo endothelial cells with the biological sample from step (a);

(c) assessing levels of C5b-9 deposition on the cells; and

(d) normalizing levels of C5b-9 deposition by cell number,

wherein less C5b-9 deposition with the biological sample incubated with eculizumab compared to without eculizumab indicates the patient is likely to benefit from treatment with eculizumab.

In some embodiments, the complement-associated disorder is aHUS. Accordingly, provided herein are methods for determining whether a patient with atypical hemolytic uremic syndrome (aHUS) is likely to benefit from treatment with eculizumab, the method comprising:

(a) incubating a biological sample obtained from the patient with and without eculizumab;

(b) contacting ex vivo endothelial cells with the biological sample from step (a);

(c) assessing levels of C5b-9 deposition on the cells; and

(d) normalizing levels of C5b-9 deposition by cell number, wherein less C5b-9 deposition with the biological sample incubated with eculizumab compared to without eculizumab indicates the patient is likely to benefit from treatment with eculizumab.

Once a patient with or suspected of having a complement-associated disorder is identified as being likely to benefit from treatment with an inhibitor of C5 (e.g., eculizumab) using the methods described herein, the patient can be treated with a therapeutic inhibitor of C5, such as the inhibitor used in the assay (e.g., eculizumab).

Accordingly, provided herein are methods of treating a patient with or suspected of having a complement-associated disorder, as determined by the level of C5b-9 deposition on a cell type relevant to the disorder or disease (e.g., endothelial cells for aHUS) using the methods disclosed herein, comprising administering to the patient a therapeutically-effective amount of an inhibitor of C5, e.g., eculizumab, or any of the inhibitors of C5 described in the next section. Details regarding administering inhibitors of C5 to patients with complement-associated disorders can be found, e.g., in WO2010054403 and WO2015/021166, the contents of which are herein incorporated by reference in their entirety.

For patients who have a complement-associated disorder and are undergoing treatment with an inhibitor of C5, also provided herein are methods for monitoring the efficacy of treatment by, for example, determining whether the dosage of inhibitor being administered to the patient is sufficient to normalize C5b-9 deposition on endothelial cells in the ex vivo assays described herein. According, provided herein are methods for monitoring the efficacy of treatment of a patient who has a complement-associated disorder and is being treated with an inhibitor of C5, the method comprising:

(a) contacting ex vivo endothelial cells with a biological sample from the patient and a control sample;

(b) assessing levels of C5b-9 deposition on the cells;

(c) normalizing levels of C5b-9 deposition by cell number; and

(d) increasing the dose of the inhibitor administered to the patient if C5b-9 deposition with the biological sample from the patient being treated with the inhibitor is greater compared to C5b-9 deposition with the control sample. In some embodiments, if the patient is administered an increased dose of the inhibitor based on the results of step (d), steps (a)-(c) are repeated to determine whether the increased dose is sufficient to normalize levels of C5b-9 deposition on the cells. These steps can be repeated until a dosage sufficient to normalize levels of C5b-9 deposition is determined. Normalized levels of C5b-9 deposition on cells refer to levels of C5b-9 deposition essentially similar to C5b-9 deposition observed with a controls sample. In some embodiments, normalized levels relative to that of the control sample may indicate a level of C5b-9 lower than that of the control, or, if higher, about 1-20% higher than that of the control, such as about 2%, about 3%, about 4%, about 5%, about 6%, about 7%, about 8%, about 9%, about 10%, about 11%, about 12%, about 13%, about 14%, about 15%, about 16%, about 17%, about 18%, about 19% or about 20% higher.

Also provided herein are methods for screening candidate inhibitors of C5 comprising:

(a) incubating a biological sample from a patient known to have a complement-associated disorder with and without the candidate inhibitor;

(b) contacting ex vivo endothelial cells with the biological sample from step (a);

(c) assessing levels of C5b-9 deposition on the cells; and

(d) normalizing levels of C5b-9 deposition by cell number,

wherein less C5b-9 deposition with the biological sample incubated with the candidate inhibitor compared to without the inhibitor indicates the candidate inhibitor has anti-C5 activity. Such methods can be performed in parallel with a positive control, e.g., an inhibitor of C5 with known and validated inhibitory activity.

Exemplary inhibitors of C5 that are suitable for use in the methods described herein are described in the next section.

### IV. Inhibitors of Human Complement Component C5

Suitable inhibitors of human complement component C5 (“inhibitors of C5”) for use in the methods described herein can include any inhibitor be any molecule that binds to or otherwise blocks the generation of C5b-9 and/or activity of C5. For example, the “inhibitor of C5” can be any agent that inhibits: (i) the expression, or proper intracellular trafficking or secretion by a cell, of a complement component C5 protein; (ii) the activity of C5 cleavage fragments C5a or C5b (e.g., the binding of C5a to its cognate cellular receptors or the binding of C5b to C6 and/or other components of the terminal complement complex; see above); (iii) the cleavage of a human C5 protein to form C5a and C5b; (iv) the proper intracellular trafficking of, or secretion by a cell, of a complement component C5 protein; or (v) the stability of C5 protein or the mRNA encoding C5 protein. Inhibition of complement component C5 protein expression includes: inhibition of transcription of a gene encoding a human C5 protein; increased degradation of an mRNA encoding a human C5 protein; inhibition of translation of an mRNA encoding a human C5 protein; increased degradation of a human C5 protein; inhibition of proper processing of a pre-pro human C5 protein; or inhibition of proper trafficking or secretion by a cell of a human C5 protein. Methods for determining whether a candidate agent is an inhibitor of human complement component C5 are known in the art and described herein. Any complement inhibitor that prevents the formation or induces the decay of C3 convertase and/or C5 convertase can be screened using the methods described herein.

The inhibitor also can contain naturally occurring or soluble forms of complement C5 inhibitory compounds. Other inhibitors which may be utilized to bind to or otherwise block the generation and/or activity of complement C5 such as, e.g., proteins, protein fragments, peptides, small molecules, RNA aptamers including ARC 187 (which is commercially available from Archemix Corporation, Cambridge, Mass.), L-RNA aptamers, spiegelmers, antisense compounds, serine protease inhibitors, molecules which may be utilized in RNA interference (RNAi) such as double stranded RNA including small interfering RNA (siRNA), locked nucleic acid (LNA) inhibitors, peptide nucleic acid (PNA) inhibitors, etc.

In some embodiments, the inhibitor inhibits the activation of complement. In some embodiments, the inhibitor inhibits formation or assembly of the C3 convertase and/or C5 convertase of the alternative and/or classical pathways of complement. In some embodiments, the inhibitor inhibits terminal complement formation, e.g., formation of the C5b-9 membrane attack complex. For example, an antibody complement inhibitor may include an anti-C5 antibody. Such anti-C5 antibodies may directly interact with C5 and/or C5b, so as to inhibit the formation of and/or physiologic function of C5b.

An inhibitor of C5 can be, e.g., a small molecule, a polypeptide, a polypeptide analog, a nucleic acid, or a nucleic acid analog.

“Small molecule” as used herein, is meant to refer to an agent, which has a molecular weight preferably of less than about 6 kDa and most preferably less than about 2.5 kDa. Many pharmaceutical companies have extensive libraries of chemical and/or biological mixtures comprising arrays of small molecules, often fungal, bacterial, or algal extracts, which can be screened with any of the assays of the application. This application contemplates using, among other things, small chemical libraries, peptide libraries, or collections of natural products. Tan et al. described a library with over two million synthetic compounds that is compatible with miniaturized cell-based assays (J Am Chem Soc (1998) 120:8565-8566). It is within the scope of this application that such a library may be used to screen for agents that bind to a target antigen of interest (e.g., complement component C5). There are numerous commercially available compound libraries, such as the Chembridge DIVERSet. Libraries are also available from academic investigators, such as the Diversity set from the NCI developmental therapeutics program. Rational drug design may also be employed. For example, rational drug design can employ the use of crystal or solution structural information on the human complement component C5 protein. See, e.g., the structures described in Hagemann et al. (2008) J Biol Chem 283(12):7763-75 and Zuiderweg et al. (1989) Biochemistry 28(1):172-85. Rational drug design can also be achieved based on known compounds, e.g., a known inhibitor of C5 (e.g., an antibody, or antigen-binding fragment thereof, that binds to a human complement component C5 protein).

Peptidomimetics can be compounds in which at least a portion of a subject polypeptide is modified, and the three dimensional structure of the peptidomimetic remains substantially the same as that of the subject polypeptide. Peptidomimetics may be analogues of a subject polypeptide of the disclosure that are, themselves, polypeptides containing one or more substitutions or other modifications within the subject polypeptide sequence. Alternatively, at least a portion of the subject polypeptide sequence may be replaced with a nonpeptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal. It should be noted that peptidomimetics may or may not have similar two-dimensional chemical structures, but share common three-dimensional structural features and geometry. Each peptidomimetic may further have one or more unique additional binding elements.

Nucleic acid inhibitors of C5 can be used to bind to and inhibit C5. The nucleic acid antagonist can be, e.g., an aptamer. Aptamers are short oligonucleotide sequences that can be used to recognize and specifically bind almost any molecule, including cell surface proteins. The systematic evolution of ligands by exponential enrichment (SELEX) process is powerful and can be used to readily identify such aptamers. Aptamers can be made for a wide range of proteins of importance for therapy and diagnostics, such as growth factors and cell surface antigens. These oligonucleotides bind their targets with similar affinities and specificities as antibodies do (see, e.g., Ulrich (2006) Handb Exp Pharmacol. 173:305-326).

In some embodiments, the inhibitor of C5 is a non-antibody scaffold protein. These proteins are, generally, obtained through combinatorial chemistry-based adaptation of pre-existing antigen-binding proteins. For example, the binding site of human transferrin for human transferrin receptor can be modified using combinatorial chemistry to create a diverse library of transferrin variants, some of which have acquired affinity for different antigens. Ali et al. (1999) J Biol Chem 274:24066-24073. The portion of human transferrin not involved with binding the receptor remains unchanged and serves as a scaffold, like framework regions of antibodies, to present the variant binding sites. The libraries are then screened, as an antibody library is, against a target antigen of interest to identify those variants having optimal selectivity and affinity for the target antigen. Non-antibody scaffold proteins, while similar in function to antibodies, are touted as having a number of advantages as compared to antibodies, which advantages include, among other things, enhanced solubility and tissue penetration, less costly manufacture, and ease of conjugation to other molecules of interest. Hey et al. (2005) TRENDS Biotechnol 23(10):514-522.

One of skill in the art would appreciate that the scaffold portion of the non-antibody scaffold protein can include, e.g., all or part of: the Z domain of S. aureus protein A, human transferrin, human tenth fibronectin type III domain, kunitz domain of a human trypsin inhibitor, human CTLA-4, an ankyrin repeat protein, a human lipocalin, human crystallin, human ubiquitin, or a trypsin inhibitor from E. elaterium. Id.

In some embodiments, the inhibitor of C5 is an antibody, or antigen-binding fragment thereof, which binds to a human complement component C5 protein. Anti-C5 antibodies (or VH/VL domains derived therefrom) suitable for use in the invention can be generated using methods well known in the art. Alternatively, art recognized anti-C5 antibodies can be used. Antibodies that compete with any of these art-recognized antibodies for binding to C5 also can be used.

In one embodiment, the anti-C5 antibody prevents the generation of the anaphylatoxic activity associated with C5a and/or preventing the assembly of the membrane attack complex C5b-9. In another embodiment, the anti-C5 antibodies described herein bind to complement component C5 (e.g., human C5) and inhibit the cleavage of C5 into fragments C5a and C5b. In some embodiments, the anti-C5 antibody can bind to an epitope in the alpha chain of the human complement component C5 protein. Antibodies that bind to the alpha chain of C5 are described in, for example, WO 2010/015608 and U.S. Pat. No. 6,355,245. In some embodiments, the anti-C5 antibody can bind to an epitope in the beta chain of the human complement component C5 protein. Antibodies that bind to the C5 beta chain are described in, e.g., Moongkarndi et al. (1982) Immunobiol 162:397; Moongkarndi et al. (1983) Immunobiol 165:323; and Mollnes et al. (1988) Scand J Immunol 28:307-312. In some embodiments, the anti-C5 antibody is an antibody described in U.S. Pat. No. 9,079,949, the contents of which are herein incorporated by reference.

Additional exemplary antigenic fragments of human complement component C5 are disclosed in, e.g., U.S. Pat. No. 6,355,245, the disclosure of which is incorporated herein by reference.

Additional anti-C5 antibodies, and antigen-binding fragments thereof, suitable for use in the methods described herein are described in, e.g., PCT application publication no. WO 2010/015608, the disclosure of which is incorporated herein by reference in its entirety.

In some embodiments, the anti-C5 antibody specifically binds to a human complement component C5 protein (e.g., the human C5 protein having the amino acid sequence depicted in SEQ ID NO: 1). The terms “specific binding” or “specifically binds” refer to two molecules forming a complex (e.g., a complex between an antibody and a complement component C5 protein) that is relatively stable under physiologic conditions. Typically, binding is considered specific when the association constant (Kₐ) is higher than 10⁶ M⁻¹. Thus, an antibody can specifically bind to a C5 protein with a Kₐ of at least (or greater than) 10⁶ (e.g., at least or greater than 10⁷, 10⁸, 10⁹, 10¹⁰, 10¹¹ 10¹², 10¹³, 10¹⁴, or 10¹⁵ or higher) M⁻¹. Examples of antibodies that specifically bind to a human complement component C5 protein are described in, e.g., U.S. Pat. No. 6,355,245, the disclosure of which is incorporated herein by reference in its entirety.

The anti-C5 antibodies described herein can have activity in blocking the generation or activity of the C5a and/or C5b active fragments of a complement component C5 protein (e.g., a human C5 protein). Through this blocking effect, the anti-C5 antibodies inhibit, e.g., the proinflammatory effects of C5a and the generation of the C5b-9 membrane attack complex (MAC) at the surface of a cell. Anti-C5 antibodies that have the ability to block the generation of C5a are described in, e.g., Moongkarndi et al. (1982) Immunobiol 162:397 and Moongkarndi et al. (1983) Immunobiol 165:323.

In some embodiments, an anti-C5 antibody, or antigen-binding fragment thereof, can reduce the ability of a C5 protein to bind to human complement component C3b (e.g., C3b present in an AP or CP C5 convertase complex) by greater than 50 (e.g., greater than 55, 60, 65, 70, 75, 80, 85, 90, or 95 or more) %. In some embodiments, upon binding to a C5 protein, the anti-C5 antibody or antigen-binding fragment thereof can reduce the ability of the C5 protein to bind to complement component C4b (e.g., C4b present in a CP C5 convertase) by greater than 50 (e.g., greater than 55, 60, 65, 70, 75, 80, 85, 90, or 95 or more) %. Methods for determining whether an antibody can block the generation or activity of the C5a and/or C5b active fragments of a complement component C5 protein, or binding to complement component C4b or C3b, are known in the art and described in, e.g., U.S. Pat. No. 6,355,245 and Wurzner et al. (1991) Complement Inflamm 8:328-340.

An exemplary anti-C5 antibody is eculizumab (Soliris®; Alexion Pharmaceuticals, Inc., Cheshire, Conn.), or an antibody that binds to the same epitope on C5 as or competes for binding to C5 with eculizumab (See, e.g., Kaplan (2002) Curr Opin Investig Drugs 3(7):1017-23; Hill (2005) Clin Adv Hematol Oncol 3(11):849-50; and Rother et al. (2007) Nature Biotechnology 25(11):1256-1488). Soliris®, is a formulation of eculizumab which is a recombinant humanized monoclonal IgG2/4κ antibody produced by murine myeloma cell culture and purified by standard bioprocess technology. Eculizumab contains human constant regions from human IgG2 sequences and human IgG4 sequences and murine complementarity-determining regions grafted onto the human framework light- and heavy-chain variable regions. Eculizumab is composed of two 448 amino acid heavy chains and two 214 amino acid light chains and has a molecular weight of approximately 148 kDa. Eculizumab comprises the heavy and light chain amino acid sequences set forth in SEQ ID NOs: 10 and 11, respectively; heavy and light chain variable region amino acid sequences set forth in SEQ ID NOs: 7 and 8, respectively; and heavy chain variable region CDR1-3 and light chain variable region CDR1-3 sequences set forth in SEQ ID NOs: 1, 2, and 3 and 4, 5, and 6, respectively.

Another exemplary antibody is, pexelizumab (Alexion Pharmaceuticals, Inc., Cheshire, Conn.), or an antibody that binds to the same epitope on C5 as or competes for binding to C5 with pexelizumab (See, e.g., Whiss (2002) Curr Opin Investig Drugs 3(6):870-7; Patel et al. (2005) Drugs Today (Barc) 41(3):165-70; and Thomas et al. (1996) Mol Immunol 33(17-18):1389-401).

Another exemplary anti-C5 antibody is antibody BNJ441 comprising heavy and light chains having the sequences shown in SEQ ID NOs: 14 and 11, respectively, or antigen binding fragments and variants thereof. BNJ441 (also known as ALXN1210) is described in PCT/US2015/019225 and U.S. Pat. No. 9,079,949, the teachings or which are hereby incorporated by reference. BNJ441 is a humanized monoclonal antibody that is structurally related to eculizumab (Soliris®). BNJ441 selectively binds to human complement protein C5, inhibiting its cleavage to C5a and C5b during complement activation. This inhibition prevents the release of the proinflammatory mediator C5a and the formation of the cytolytic pore-forming membrane attack complex C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of BNJ441. Accordingly, in one embodiment, the antibody comprises the CDR1, CDR2, and CDR3 domains of the VH region of BNJ441 having the sequence set forth in SEQ ID NO: 12, and the CDR1, CDR2 and CDR3 domains of the VL region of BNJ441 having the sequence set forth in SEQ ID NO: 8. In another embodiment, the antibody comprises heavy chain CDR1, CDR2 and CDR3 domains having the sequences set forth in SEQ ID NOs: 19, 18, and 3, respectively, and light chain CDR1, CDR2 and CDR3 domains having the sequences set forth in SEQ ID NOs: 4, 5, and 6, respectively. In another embodiment, the antibody comprises VH and VL regions having the amino acid sequences set forth in SEQ ID NO: 12 and SEQ ID NO:8, respectively.

Yet another exemplary anti-C5 antibody is antibody BNJ421 comprising heavy and light chains having the sequences shown in SEQ ID NOs: 20 and 11, respectively, or antigen binding fragments and variants thereof. BNJ421 (also known as ALXN1211) is described in PCT/US2015/019225 and U.S. Pat. No. 9,079,949, the teachings or which are hereby incorporated by reference.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO: 12, and the CDR1, CDR2 and CDR3 domains of the VL region of BNJ421 having the sequence set forth in SEQ ID NO: 8. In another embodiment, the antibody comprises heavy chain CDR1, CDR2 and CDR3 domains having the sequences set forth in SEQ ID NOs: 19, 18, and 3, respectively, and light chain CDR1, CDR2 and CDR3 domains having the sequences set forth in SEQ ID NOs: 4, 5, and 6, respectively. In another embodiment, the antibody comprises VH and VL regions having the amino acid sequences set forth in SEQ ID NO: 12 and SEQ ID NO: 8, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33(17/18):1389-1401] exemplifies the identification of CDR boundaries according to Kabat and Chothia definitions.

In some embodiments, an anti-C5 antibody described herein comprises a heavy chain CDR1 comprising, or consisting of, the following amino acid sequence: GHIFSNYWIQ (SEQ ID NO: 19).

In some embodiments, an anti-C5 antibody described herein comprises a heavy chain CDR2 comprising, or consisting of, the following amino acid sequence: EILPGSGHTEYTENFKD (SEQ ID NO: 18). In some embodiments, an anti-C5 antibody described herein comprises a heavy chain variable region comprising the following amino acid sequence:

(SEQ ID NO: 12)
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS.

In some embodiments, an anti-C5 antibody described herein comprises a light chain variable region comprising the following amino acid sequence:

(SEQ ID NO: 8)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. For example, the Fc constant region can comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., PCT/US2015/019225 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution described by Dall'Acqua et al. (2006) J Biol Chem 281: 23514-23524; (2) the M428L or T250Q/M428L substitutions described in Hinton et al. (2004) J Biol Chem 279:6213-6216 and Hinton et al. (2006) J Immunol 176:346-356; and (3) the N434A or T307/E380A/N434A substitutions described in Petkova et al. (2006) Int Immunol 18(12):1759-69. The additional substitution pairings: P257I/Q311I, P257I/N434H, and D376V/N434H are described in, e.g., Datta-Mannan et al. (2007) J Biol Chem 282(3):1709-1717, the disclosure of which is incorporated herein by reference in its entirety.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three, or two) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I, and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in BNJ441 and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434, or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable an anti-C5 antibodies for use in the methods described herein, in some embodiments, comprise a heavy chain polypeptide comprising the amino acid sequence depicted in SEQ ID NO: 14 and/or a light chain polypeptide comprising the amino acid sequence depicted in SEQ ID NO: 11. Alternatively, the anti-C5 antibodies for use in the methods described herein, in some embodiments, comprise a heavy chain polypeptide comprising the amino acid sequence depicted in SEQ ID NO: 20 and/or a light chain polypeptide comprising the amino acid sequence depicted in SEQ ID NO: 11.

In some embodiments, the C5 inhibitor is an antibody that binds to C5a (sometimes referred to herein as “an anti-C5a antibody”). In some embodiments, the antibody binds to C5a, but not to full-length C5. In some embodiments, the binding of an antibody to C5a can inhibit the biological activity of C5a. Methods for measuring C5a activity include, e.g., chemotaxis assays, RIAs, or ELISAs (see, e.g., Ward and Zvaifler (1971) J Clin Invest 50(3):606-16 and Wurzner et al. (1991) Complement Inflamm 8:328-340). In some embodiments, the binding of an antibody to C5a can inhibit the interaction between C5a and C5aR1. Suitable methods for detecting and/or measuring the interaction between C5a and C5aR1 (in the presence and absence of an antibody) are known in the art and described in, e.g., Mary and Boulay (1993) Eur J Haematol 51(5):282-287; Kaneko et al. (1995) Immunology 86(1):149-154; Giannini et al. (1995) J Biol Chem 270(32):19166-19172; and U.S. Patent Application Publication No. 20060160726. For example, the binding of detectably labeled (e.g., radioactively labeled) C5a to C5aR1-expressing peripheral blood mononuclear cells can be evaluated in the presence and absence of an antibody. A decrease in the amount of detectably-labeled C5a that binds to C5aR1 in the presence of the antibody, as compared to the amount of binding in the absence of the antibody, is an indication that the antibody inhibits the interaction between C5a and C5aR1. In some embodiments, the binding of an antibody to C5a can inhibit the interaction between C5a and C5L2 (see below). Methods for detecting and/or measuring the interaction between C5a and C5L2 are known in the art and described in, e.g., Ward (2009) J Mol Med 87(4):375-378 and Chen et al. (2007) Nature 446(7132):203-207 (see below).

An exemplary anti-C5a antibody is antibody BNJ383 comprising heavy and light chains having the sequences shown in SEQ ID NOs: 26 and 21, respectively, or antigen binding fragments and variants thereof. BNJ383 (also known as ALXN1007) is described in WO 2011/137395 and U.S. Pat. No. 9,011,852, the teachings or which are hereby incorporated by reference. In one embodiment, the anti-C5a antibody comprises the heavy and light chain CDRs or variable regions of BNJ383. Accordingly, in one embodiment, the antibody comprises the CDR1, CDR2, and CDR3 domains of the VH region of BNJ383 having the sequence set forth in SEQ ID NO: 27, and the CDR1, CDR2 and CDR3 domains of the VL region of BNJ383 having the sequence set forth in SEQ ID NO: 22. In another embodiment, the antibody comprises heavy chain CDR1, CDR2 and CDR3 domains having the sequences set forth in SEQ ID NOs: 28, 29, and 30, respectively, and light chain CDR1, CDR2 and CDR3 domains having the sequences set forth in SEQ ID NOs: 23, 24, and 25 respectively. In another embodiment, the antibody comprises VH and VL regions having the amino acid sequences set forth in SEQ ID NO: 27 and SEQ ID NO: 22, respectively.

In some embodiments, the C5 inhibitor is an antibody that binds to C5b (sometimes referred to herein as “an anti-C5b antibody”). In some embodiments, the antibody binds to C5b, but does not bind to full-length C5. The structure of C5b is described in, e.g., Miiller-Eberhard (1985) Biochem Soc Symp 50:235-246; and Yamamoto and Gewurz (1978) J Immunol 120(6):2008-2015. As described above, C5b combines with C6, C7, and C8 to form the C5b-8 complex at the surface of the target cell. Protein complex intermediates formed during the series of combinations include C5b-6 (including C5b and C6), C5b-7 (including C5b, C6, and C7), and C5b-8 (including C5b, C6, C7, and C8). Upon binding of several C9 molecules, the membrane attack complex (MAC, C5b-9 terminal complement complex (TCC)) is formed. When sufficient numbers of MACs insert into target cell membranes, the openings they create (MAC pores) mediate rapid osmotic lysis of the target cells.

In some embodiments, the binding of an antibody to C5b can inhibit the interaction between C5b and C6. In some embodiments, the binding of the antibody to C5b can inhibit the assembly or activity of the C5b-9 MAC-TCC. In some embodiments, the binding of an antibody to C5b can inhibit complement-dependent cell lysis (e.g., in vitro and/or in vivo). Suitable methods for evaluating whether an antibody inhibits complement-dependent lysis include, e.g., hemolytic assays or other functional assays for detecting the activity of soluble C5b-9. For example, a reduction in the cell-lysing ability of complement in the presence of an antibody can be measured by a hemolysis assay described by Kabat and Mayer (eds.), “Experimental Immunochemistry, 2^{nd} Edition,” 135-240, Springfield, Ill., CC Thomas (1961), pages 135-139, or a conventional variation of that assay such as the chicken erythrocyte hemolysis method as described in, e.g., Hillmen et al. (2004) N Engl J Med 350(6):552.

Antibodies that bind to C5b as well as methods for making such antibodies are known in the art. Commercially available anti-C5b antibodies are available from a number of vendors including, e.g., Hycult Biotechnology (catalogue number: HM2080; clone 568) and Abcam™ (ab46151 or ab46168).

Antibodies, or antigen-binding fragments thereof, suitable for use in the methods described herein can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246: 1275-1281 (1989).

Methods for determining whether a particular agent is an inhibitor of human complement component C5 are described herein and are known in the art. For example, the concentration and/or physiologic activity of C5a and C5b in a body fluid can be measured by methods well known in the art. Methods for measuring C5a concentration or activity include, e.g., chemotaxis assays, RIAs, or ELISAs (see, e.g., Ward and Zvaifler (1971) J Clin Invest. 50(3):606-16 and Wurzner et al. (1991) Complement Inflamm. 8:328-340). For C5b, hemolytic assays or assays for soluble C5b-9 as discussed herein can be used. Other assays known in the art can also be used. Using assays of these or other suitable types, candidate agents capable of inhibiting human complement component C5 such as an anti-C5 antibody, can be screened in order to, e.g., identify compounds that are useful in the methods described herein and determine the appropriate dosage levels of such compounds.

Methods for determining whether a candidate compound inhibits the cleavage of human C5 into forms C5a and C5b are known in the art and described in, e.g., Moongkarndi et al. (1982) Immunobiol 162:397; Moongkarndi et al. (1983) Immunobiol 165:323; Isenman et al. (1980) J Immunol 124(1):326-31; Thomas et al. (1996) Mol. Immunol 33(17-18):1389-401; and Evans et al. (1995) Mol. Immunol 32(16):1183-95. Moreover, the methods described herein can be used to screen for candidate inhibitors of C5, i.e., screening for molecules that inhibit C5b-9 deposition on cells (e.g., endothelial cells) ex vivo.

Inhibition of human complement component C5 can also reduce the cell-lysing ability of complement in a subject's body fluids. Such reductions of the cell-lysing ability of complement present can be measured by methods well known in the art such as, for example, by a conventional hemolytic assay such as the hemolysis assay described by Kabat and Mayer (eds), “Experimental Immunochemistry, 2^{nd} Edition,” 135-240, Springfield, Ill., CC Thomas (1961), pages 135-139, or a conventional variation of that assay such as the chicken erythrocyte hemolysis method as described in, e.g., Hillmen et al. (2004) N Engl J Med 350(6):552.

### V. Diagnostic Kits

Also provided herein are kits which include the components for carrying out the methods described herein and instructions for use. Accordingly, in some embodiments, the kit comprises cells relevant to the complement-associated disorder or disease of interest, an anti-C5b-9 antibody, and a means for detecting the anti-C5b-9 antibody, such as a secondary antibody comprising a detectable moiety. Such kits may comprise at least one additional reagent, such as buffers, stabilizers, substrates, immunodetection reagents (primary and secondary antibodies), and/or cofactors required to perform the methods. In some embodiments, the kit comprises a means for collecting a biological sample from patients. Such means can comprise, for example, reagents or containers that can be used to obtain fluid or tissue samples from the patient. The kit may also comprise instructions for automating the assay, e.g., by providing guidance on how to use the methods in conjunction with commercially-available automated platforms (e.g., LI-COR Odyssey CLX platform).

Exemplary methods and materials are described below, although methods and materials similar or equivalent to those described herein can also be used in the practice or testing of the presently disclosed methods and compositions. All publications, patent applications, patents, and other references mentioned herein are incorporated by reference in their entirety. In particular, the disclosures of PCT Application Nos. PCT/US2009/063929 (WO2010/054403) and PCT/US2014/049957 (WO2015/021166) are expressly incorporated herein by reference.

### VI. Exemplary Embodiments

1. A method for measuring complement C5b-9 deposition comprising:

(a) contacting ex vivo a biological sample obtained from a patient who has or is suspected of having a complement-associated disorder with disease-relevant cells;

(b) assessing levels of C5b-9 deposition on the cells;

(c) normalizing levels of C5b-9 deposition by cell number.

2. A method for determining whether a patient with a complement-associated disorder would benefit from treatment with an inhibitor of C5, the method comprising:

(a) incubating a biological sample obtained from the patient with and without an inhibitor of C5;

(b) contacting ex vivo endothelial cells with the biological sample from step (a);

(c) assessing levels of C5b-9 deposition on the cells; and

(d) normalizing levels of C5b-9 deposition by cell number, wherein less C5b-9 deposition with the biological sample incubated with the inhibitor compared to without the inhibitor indicates the patient is likely to benefit from treatment with the inhibitor.

3. A method for determining whether a patient with a complement-associated disorder is likely to benefit from treatment with eculizumab, the method comprising:

(a) incubating a biological sample obtained from the patient with and without eculizumab;

(b) contacting ex vivo endothelial cells with the biological sample from step (a);

(c) assessing levels of C5b-9 deposition on the cells; and

(d) normalizing levels of C5b-9 deposition by cell number, wherein less C5b-9 deposition with the biological sample incubated with eculizumab compared to without eculizumab indicates the patient is likely to benefit from treatment with eculizumab.

4. A method for determining whether a patient with atypical hemolytic uremic syndrome (aHUS) is likely to benefit from treatment with eculizumab, the method comprising:

(a) incubating a biological sample obtained from the patient with and without eculizumab;

(b) contacting ex vivo endothelial cells with the biological sample from step (a);

(c) assessing levels of C5b-9 deposition on the cells; and

(d) normalizing levels of C5b-9 deposition by cell number, wherein less C5b-9 deposition with the biological sample incubated with eculizumab compared to without eculizumab indicates the patient is likely to benefit from treatment with eculizumab.

5. A method for monitoring a patient who has a complement-associated disorder and is being treated with an inhibitor of C5, the method comprising:

(a) contacting ex vivo endothelial cells with a biological sample from the patient and a control sample;

(b) assessing levels of C5b-9 deposition on the cells;

(c) normalizing levels of C5b-9 deposition by cell number; and

(d) increasing the dose of the inhibitor administered to the patient if C5b-9 deposition with the biological sample from the patient being treated with the inhibitor is greater compared to C5b-9 deposition with the control sample.

6. The method of embodiment 5, wherein if the patient is administered an increased dose of the inhibitor, steps (a)-(c) are repeated to determine whether the increased dose is sufficient to normalize levels of C5b-9 deposition on the cells.
7. A method of treating a complement-associated disorder in a patient determined to be responsive to an inhibitor of C5 or eculizumab according to the method of any one of embodiments 1-4, the method comprising administering to the patient a therapeutically-effective amount of the inhibitor or eculizumab.
8. The method of embodiment 2, 5, or 6, wherein the inhibitor of C5 is an antibody, such as eculizumab.
9. The method of any one of the preceding embodiments, wherein the cells are cultured on a solid platform, such as a microplate.
10. The method of embodiment 9, wherein the solid platform is a 96-well microplate.
11. The method of any one of the preceding embodiments, wherein the disease-relevant cells are selected from the group consisting of endothelial cells, retinal pigment epithelial cells, chondrocytes, neurons, glial cells, skeletal muscle cells, and cardiomyocytes.
12. The method of embodiment 11, wherein the disease-relevant cells are endothelial cells selected from the group consisting of human microvascular endothelial cells from dermal origin, human umbilical vein endothelial cells, endothelial cells from foreskin, and endothelial cells from liver adenocarcinoma.
13. The method of any one of the preceding embodiments, wherein the cells are plated at a density of about 5,000 to about 6,000 cells per well and cultured until confluent.
14. The method of any one of the preceding embodiments, wherein the cells are plated at a density of about 10,000 cells to about 12,500 cells per well and cultured until confluent.
15. The method of any one of embodiments 1-12, wherein the cells are plated at a density of about 15,000 cells per well cultured until confluent.
16. The method of any one of the preceding embodiments, wherein cells are confluent before being contacted with the biological sample.
17. The method of any one of the preceding embodiments, wherein the biological sample is serum.
18. The method of embodiment 17, wherein the serum is from a patient with aHUS, a patient in remission, or an eculizumab-naïve patient.
19. The method of any one of the preceding embodiments, wherein the cells are activated with adenosine 5′-diphosphate, thrombin, or lipopolysaccharide.
20. The method of any one of the preceding embodiments, wherein the cells are contacted with the biological sample for about 1.5 hours to about 4 hours.
21. The method of any one of the preceding embodiments, wherein the cells are incubated with a fixative such as paraformaldehyde after the contacting step but before the assessing step.
22. The method of any one of the preceding embodiments, wherein the levels of C5b-9 deposition are assessed using an anti-C5b-9 antibody.
23. The method of embodiment 20, wherein the anti-C5b-9 antibody is detected with a secondary antibody comprising a detectable label such as a dye.
24. The method of any one of the preceding embodiments, wherein the levels of C5b-9 deposition are assessed using an On-cell Western assay.
25. The method of embodiment 23, wherein the cells are permeabilized after the anti-C5b-9 antibody is detected with the secondary antibody.
26. The method of embodiment 23, wherein the cells are permeabilized before the anti-C5b-9 antibody is detected with the secondary antibody.
27. The method of embodiment 25 or 26, wherein, following permeabilization, the cells are incubated with an agent that accumulates in the nucleus, such as an agent that stains DNA.
28. The method of embodiment 27, wherein the agent is selected from the group consisting of: CellTag 700 Stain, DAPI, acridine orange, Hoechst 33342 Dye, Hoechst 33258, SYTOX Green nucleic acid stain, and Vybrant DyeCycle stain.
29. The method of any one of the preceding embodiments, wherein one or more steps are automated.
30. The method of any one of embodiments 1-3, 5-17, and 19-29, wherein the patient has atypical hemolytic uremic syndrome, STEC-HUS, diabetes, lupus nephritis, vasculitis, or chronic allograft rejection.
31. A method for measuring complement C5b-9 deposition comprising:

(a) contacting ex vivo a biological sample obtained from a patient who has or is suspected of having a complement-associated disorder with disease-relevant cells;

(b) assessing levels of C5b-9 deposition on the cells;

(c) permeabilizing the cells before or after assessing levels of C5b-9 on the cells; and

(c) normalizing levels of C5b-9 deposition by cell number.

32. A method for determining whether a patient with a complement-associated disorder would benefit from treatment with an inhibitor of C5, the method comprising:

(a) incubating a biological sample obtained from the patient with and without an inhibitor of C5;

(b) contacting ex vivo endothelial cells with the biological sample from step (a);

(c) assessing levels of C5b-9 deposition on the cells;

(d) permeabilizing the cells before or after assessing levels of C5b-9 on the cells; and

(e) normalizing levels of C5b-9 deposition by cell number, wherein less C5b-9 deposition with the biological sample incubated with the inhibitor compared to without the inhibitor indicates the patient is likely to benefit from treatment with the inhibitor.

33. A method for determining whether a patient with a complement-associated disorder is likely to benefit from treatment with eculizumab, the method comprising:

(a) incubating a biological sample obtained from the patient with and without eculizumab;

(b) contacting ex vivo endothelial cells with the biological sample from step (a);

(c) assessing levels of C5b-9 deposition on the cells;

(d) permeabilizing the cells before or after assessing levels of C5b-9 on the cells; and

(e) normalizing levels of C5b-9 deposition by cell number, wherein less C5b-9 deposition with the biological sample incubated with eculizumab compared to without eculizumab indicates the patient is likely to benefit from treatment with eculizumab.

34. A method for determining whether a patient with atypical hemolytic uremic syndrome (aHUS) is likely to benefit from treatment with eculizumab, the method comprising:

(a) incubating a biological sample obtained from the patient with and without eculizumab;

(b) contacting ex vivo endothelial cells with the biological sample from step (a);

(c) assessing levels of C5b-9 deposition on the cells;

(d) permeabilizing the cells before or after assessing levels of C5b-9 on the cells; and

(e) normalizing levels of C5b-9 deposition by cell number, wherein less C5b-9 deposition with the biological sample incubated with eculizumab compared to without eculizumab indicates the patient is likely to benefit from treatment with eculizumab.

35. A method for monitoring a patient who has a complement-associated disorder and is being treated with an inhibitor of C5, the method comprising:

(a) contacting ex vivo endothelial cells with a biological sample from the patient and a control sample;

(b) assessing levels of C5b-9 deposition on the cells;

(c) permeabilizing the cells before or after assessing levels of C5b-9 on the cells;

(d) normalizing levels of C5b-9 deposition by cell number; and

(e) increasing the dose of the inhibitor administered to the patient if C5b-9 deposition with the biological sample from the patient being treated with the inhibitor is greater compared to C5b-9 deposition with the control sample.

### EXAMPLES

### Example 1: Determination of HMEC-1 Culture Conditions on Microplates

The experiments described below were conducted with the aim of automating an assay for detecting C5b-9 deposition on endothelial cells.

In this first experiment, conditions were optimized to transition the culture of endothelial cells (HMEC-1) from glass support (i.e., glass coverslips) used in the “classic method” described by Noris et al. (Blood 2014; 124:1715-26) to plastic 96-well microplates.

A human microvascular endothelial cell line of dermal origin (HMEC-1) was cultured in growth medium consisting of MCDB 131 (Gibco, Grand Island, N.Y.) supplemented with 10% fetal bovine serum (Gibco), 10 ag/ml hydrocortisone f.c., 100 U/ml penicillin f.c., 100 ag/ml streptomycin f.c., 2 mM glutamine f.c. (Gibco), and 50 ag/ml endothelial cell growth factor f.c.

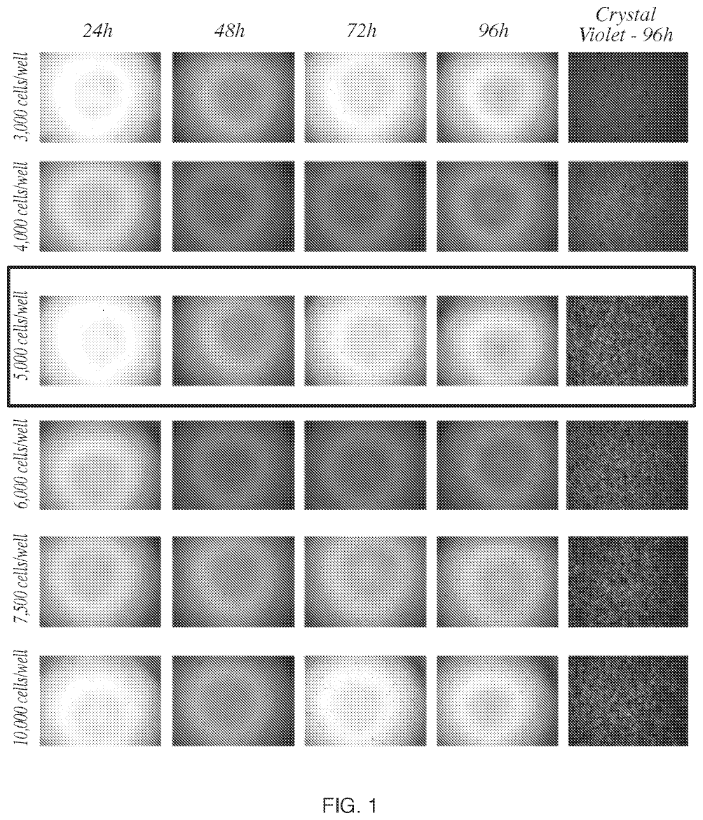

Initially, culture conditions were determined for timing the period from the seeding of cells to confluence in 96 hours using 96-well microplates. Specifically, cells were seeded at 3,000, 4,000, 5,000, 6,000, 7,500, or 10,000 cells per well in 96-well microplates. Cells were observed using a phase contrast microscope after 24, 48, 72, and 96 hours of culture in growth medium. In three independent experiments, seeding at 5,000 cells per well achieved a confluent monolayer at 96 hours (FIG. 1). Indeed, wells seeded with 3,000 and 4,000 HMEC-1 cells at 96 hours did not reach confluence. When seeded at 7,500 or 10,000 cells per well, HMEC-1 cells formed multiple layers. Confluence was obtained at 96 hours in wells seeded with 5,000 or 6,000 cells.

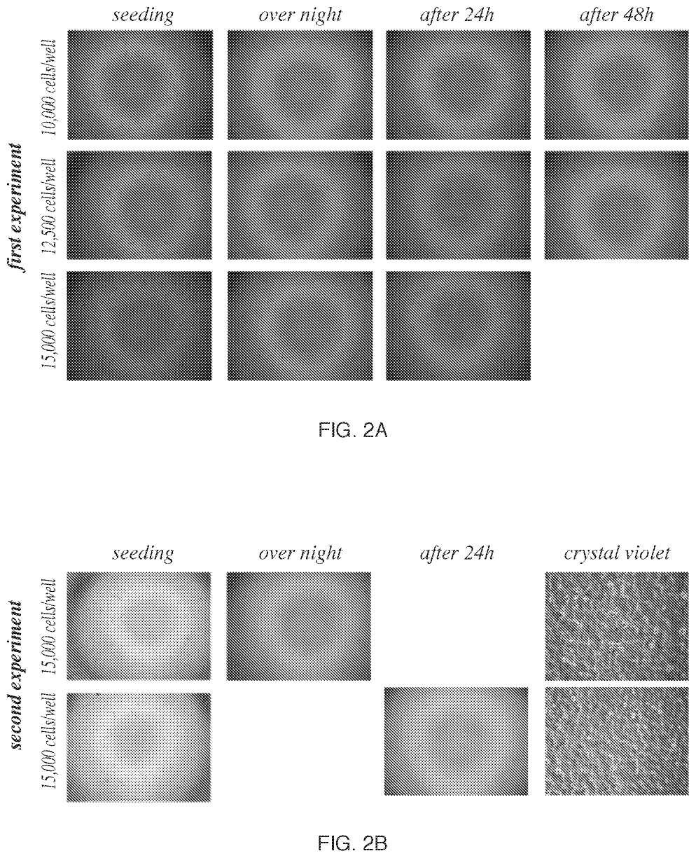

Further conditions were tested to reduce the time needed from seeding cells to confluence. Specifically, 10,000, 12,500, and 15,000 cells per well were seeded in 96-well microplates, and the growing cells were observed with a phase contrast microscope after overnight, 24, and 48 hours of culture in growth medium. In wells seeded with 10,000 and 12,500 cells, confluence was reached after 48 hours (FIG. 2A). When seeded at 15,000 per well, a confluent monolayer was reproducibly present after overnight and after 24 hours of culture (FIGS. 2A and 2B).

Next, the rate of growth of HMEC-1 cells on plastic microplates of different sizes (96-, 24-, 12- and 6-well/microplate) in growth medium was determined. HMEC-1 cells (15,625 cells/cm²) were seeded and cultured for 96 hours. At the end, adhering cells were detached by trypsinization and counted twice. Trypan blue was added to evaluate dead cells. As shown in Table 1, reproducible growth of cells was achieved in 96-, 24-, 12- and 6-well/microplates, such that the final number of cells was proportional to the number of cells seeded and to the well surface. Trypan blue exclusion showed a negligible number of dead cells within the monolayers.

TABLE 1
Mean of the results of two culture experiments
on 96-, 24-, 12- and 6- well/microplates.

| | | | number of | |
|---|---|---|---|---|
| | number | number of | cells at | population |
| | cells/cm² | cells seeding | confluence | doubling |
| 96 multiwell | 15,625 | 5,000 | 56,250 | 3.49 |
| 24 multiwell | 15,625 | 30,000 | 305,000 | 3.35 |
| 12 multiwell | 15,625 | 60,000 | 657,000 | 3.45 |
| 6 multiwell | 15,625 | 150,000 | 1300,000 | 3.11 |

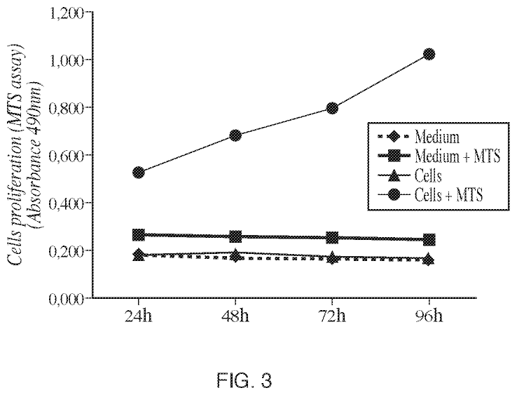

The rate of growth of 5,000 HMEC-1 cells per well seeded on 96-well microplates was also determined. Cells were seeded at 5,000 cells per well and cultured for 24, 48, 72 and 96 hours in separate plates. Cells were maintained in growth medium with the exception of those in the 96-hour plates, which were cultured for 72 hours in growth medium and then shifted to medium without serum for the last 24 hours to reproduce conditions used in the “classic” assay. At 24, 48, 72, and 96 hours, MTS reagent [3-(4,5-dimethylthiazol-2-yl)-5-(3-carboxymethoxyphenyl)-2-(4-sulphophenyl)-2H-tetrazolium, inner salt] was added for an additional 3 hours and this was followed by a reading of absorbance at 490 nm with a microplate spectrophotometer reader. As shown in FIG. 3, cells grew well at each time point analyzed.

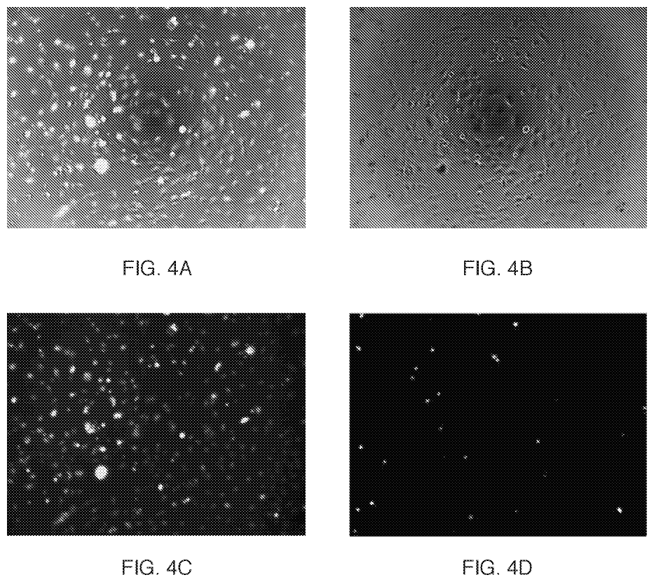

To further establish whether the cells grown on 96-well microplates were viable, 5,000 HMEC-1 were seeded on microplates and cultured for 96 hours. At the end of the culture period, medium was removed and acrydine orange and propidium iodide in PBS were added to the cells. Acridine orange (AO) and propidium iodide (PI) are nucleic acid binding dyes that can be used to measure cell viability. Since AO is cell permeable, all stained nucleated cells generate a green fluorescence. PI only enters cells with compromised membranes, and therefore dying, dead, and necrotic nucleated cells stained with PI generate a red fluorescence. Red and green cells were counted and the percentage of dead red cells was calculated. As shown in FIG. 4 and Table 2, in three independent experiments, the large majority of cells were alive.

TABLE 2
Cell numbers per high power field and percentages of dying cells after
96 hours of culture (mean ± SD of n = 3 experiments).

| no of cells | % dead cells |
|---|---|
| 164 ± 7 | 17 ± 1 |
| 152 ± 12 | 14 ± 1 |
| 143 ± 6 | 14 ± 1 |

### Example 2: Evaluation of the Effect of Incubation with Control Serum on HMEC-1 Cells

In this experiment, the effects of activating HMEC-1 cells with ADP or thrombin on cell number and viability were assessed. 5,000 HMEC-1 cells were seeded on 96-well microplates and were cultured for 96 hours (for 72 with growth medium and for the last 24 hours with medium without serum). HMEC-1 cell monolayers were washed three times with test medium (HBSS: 137 mmol/l NaCl, 5.4 mmol/l KCl, 0.7 mmol/l Na₂HPO₄, 0.73 mmol/l KH₂PO₄, 1.9 mmol/l CaCl₂, 0.8 mmol/l MgSO₄, 28 mmol/l Trizma base pH 7.3, 0.1% dextrose; with 0.5% BSA) and then activated with 10 μM ADP (Sigma-Aldrich) in test medium for 10 minutes (ADP-activated cells) or with thrombin (2 U/ml, 10 min), or incubated with test medium alone (resting cells). Resting cells or cells preactivated with ADP or thrombin were incubated for 4 hours with 50% control serum (in HBSS, 75 μL final volume). At the end of the incubation, the number of adhering cells and viability was assessed using the LIVE/DEAD cell viability assay.

As shown in Table 3, in two independent experiments, the total number of cells was comparable for all conditions and the percentages of dying cells were low and comparable to the percentages observed with medium alone.

TABLE 3
Cell numbers per high power field and percentages
of dying cells after 4 hours incubation with control
serum (mean ± SD of three wells each).

| | | |
|---|---|---|
| | Control serum | |
| resting | ADP-activated | THR-activated |

| no of | % dead | no of | % dead | no of | % dead |
|---|---|---|---|---|---|
| cells | cells | cells | cells | cells | cells |
| 157 ± 7 | 16 ± 1 | 123 ± 5 | 9 ± 1 | — | — |
| 114 ± 2 | 17 ± 7 | 108 ± 8 | 12 ± 2 | 116 ± 9 | 11 ± 2 |

Similar experiments were conducted at different cell densities. HMEC-1 cells were seeded at 10,000 and 12,500 cells per well and cultured for 48 hours on 96-well microplates. Cells seeded at 15,000 cells per well were cultured overnight or for 24 hours. Confluent HMEC-1 cells were washed three times with test medium (HBSS: 137 mmol/l NaCl, 5.4 mmol/1 KCl, 0.7 mmol/l Na₂HPO₄, 0.73 mmol/l KH₂PO₄, 1.9 mmol/l CaCl₂, 0.8 mmol/l MgSO₄, 28 mmol/1 Trizma base pH 7.3, 0.1% dextrose; with 0.5% BSA) and then activated with 10 μM ADP (Sigma-Aldrich) in test medium for 10 minutes (ADP-activated cells) or with thrombin (2 U/ml, 10 min) or incubated with test medium alone (resting cells).

Resting cells or cells pre-activated with ADP or thrombin were incubated for 4 hours with 50% control serum (in HBSS, 75 microliters final volume). At the end of the incubation we have verified cell viability by adding acrydine orange and propidium iodide to perform the LIVE/DEAD cell viability assay.

As shown in Table 4 (10,000 and 12,500 cells/well), Table 5 (15,000 cells/well), and Table 6 (15,000 cells/well duplicate experiment), the large majority of cells were alive.

TABLE 4

| first | seeding: 12,500 48 h | | seeding: 10,000 48 h | |
|---|---|---|---|---|
| experiment | total | % dead | total | % dead |
| resting | 123 ± 7 | 10.4 ± 1.1 | 167 ± 6 | 24.2 ± 1.4 |
| resting + HS | 118 ± 3 | 12.8 ± 1.4 | 173 ± 5 | 30.8 ± 1.3 |
| ADP + HS | 122 ± 5 | 11.7 ± 1.3 | 152 ± 7 | 25.2 ± 1.9 |
| THR + HS | 123 ± 5 | 11.4 ± 0.4 | 145 ± 3 | 25.4 ± 1.7 |

TABLE 5

| | seeding: 15,000 cells | |
|---|---|---|
| first | over night | 24 h |

| experiment | total | % dead | total | % dead |
|---|---|---|---|---|
| resting | 180 ± 7 | 17.6 ± 1.5 | 130 ± 11 | 17.8 ± 1.5 |
| resting + HS | 162 ± 6 | 17.6 ± 0.9 | 161 ± 7 | 22.3 ± 1.2 |
| ADP + HS | 151 ± 4 | 13.7 ± 0.8 | 147 ± 8 | 19.2 ± 1.6 |
| THR + HS | 165 ± 8 | 17.3 ± 1.1 | 138 ± 5 | 17.0 ± 1.3 |

TABLE 6

| | seeding: 15,000 cells | | | |
|---|---|---|---|---|
| second | over night | | 24 h | |
| experiment | total | % dead | total | % dead |
| resting | 199 ± 13 | 22.6 ± 1.9 | 107 ± 11 | 12.2 ± 0.8 |
| resting + HS | 210 ± 14 | 24.3 ± 1.3 | 117 ± 4 | 13.2 ± 0.7 |
| ADP + HS | 120 ± 3 | 10.6 ± 0.7 | 114 ± 5 | 13.6 ± 0.8 |
| THR + HS | 130 ± 4 | 13.4 ± 0.8 | 99 ± 5 | 14.5 ± 0.8 |

### Example 3: Evaluation of Serum-Induced C5b-9 Deposits with Patient-Derived Samples and 96 Hours Cell Culture

Using the optimal culture conditions determined in the Examples above, a pilot study was conducted using the Odyssey CLX scanner with the purpose of evaluating whether the platform could be used to develop a new automated assay of C5b-9 deposition on HMEC-1 cells.

HMEC-1 cells were seeded at 5,000 per well in 96-well microplates and used 96 hours after seeding. HMEC-1 monolayers were washed three times with test medium (HBSS: 137 mmol/l NaCl, 5.4 mmol/l KCl, 0.7 mmol/l Na₂HPO₄, 0.73 mmol/l KH₂PO₄, 1.9 mmol/l CaCl₂, 0.8 mmol/l MgSO₄, 28 mmol/l Trizma base pH 7.3, 0.1% dextrose; with 0.5% BSA) and then activated with 10 μM ADP (Sigma-Aldrich) in test medium for 10 minutes (ADP-activated cells) or incubated with test medium alone (resting cells). Cells were then washed three times with test medium and incubated for 4 hours with serum from control or from two different patients with aHUS (taken during the acute phase of the disease) diluted 1:2 with test medium (final volume 75 μL) in the presence or absence of sCR1 (a general complement inhibitor). At the end of the incubation step, cells were washed twice with PBS, fixed in 3% paraformaldehyde, then washed again twice with PBS.

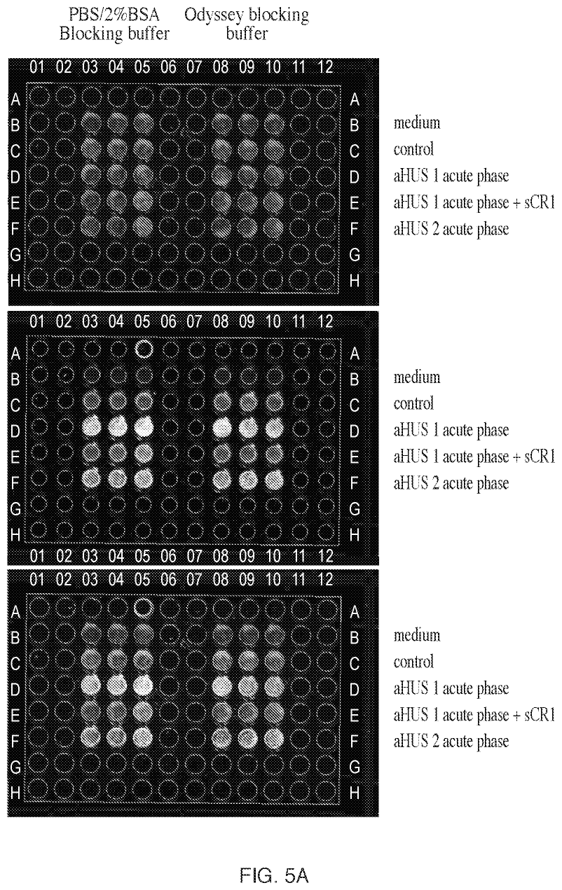

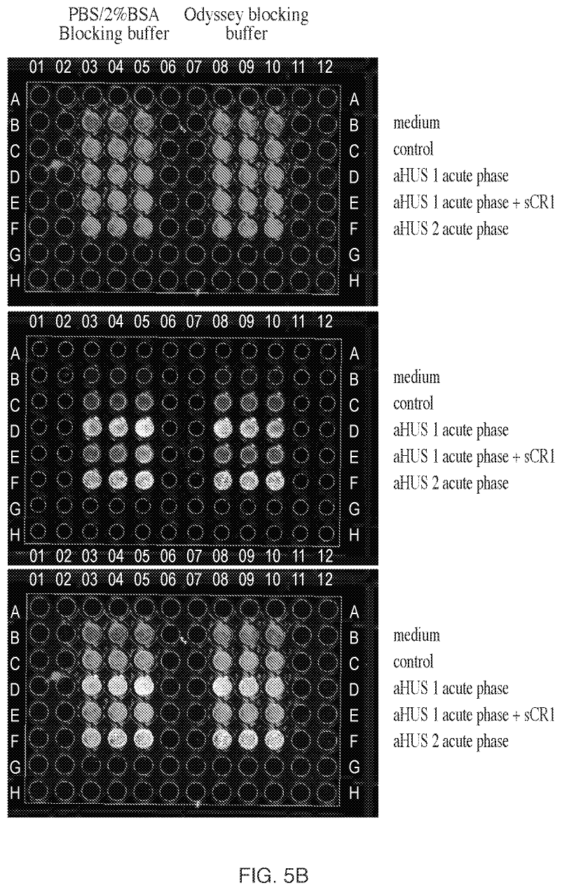

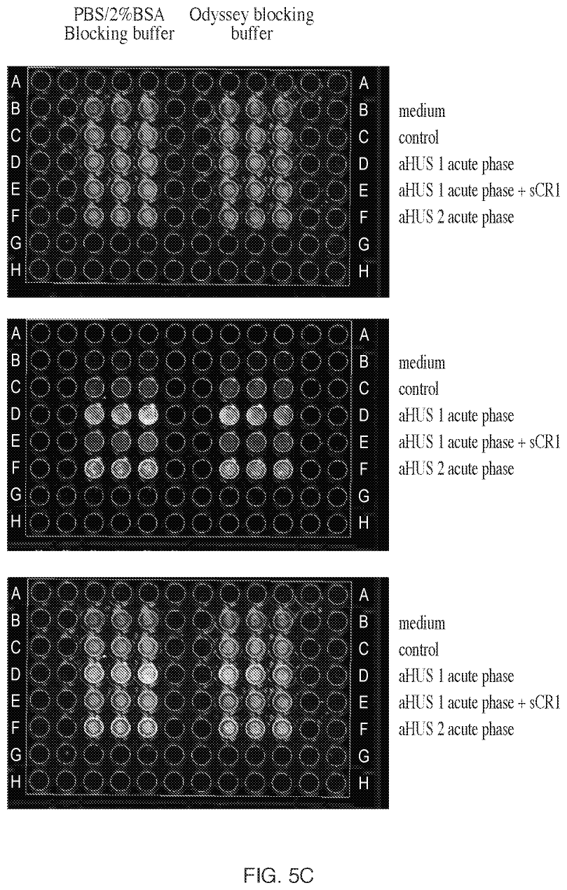

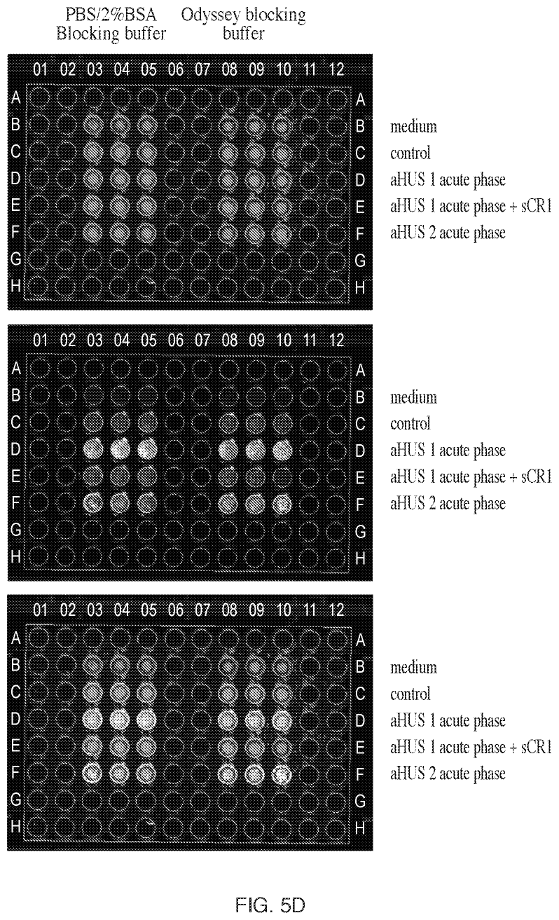

Cells were blocked for one hour with 100 μL of PBS with 2% BSA (i.e., the blocking buffer used in the “classic” assay), or with 100 μL of the commercial blocking buffer suggested by the Odyssey on-cell western protocol (Odyssey blocking buffer, LI-COR). The two blocking buffers gave comparably good results (FIGS. 5A-5D). Cells were stained with rabbit anti-human complement C5b-9 complex (Calbiochem) followed by the secondary antibody IRDye 800 CW goat anti-rabbit IgG (H+L) (LI-COR). Two different dilutions 1:600 and 1:1200 of the secondary antibody were tested.

To normalize the fluorescence intensity with cell number, after the acquisition of secondary antibody staining, cells were permeabilized with PBS 1×+0.1% Triton X-100 and then challenged with CellTag 700 Stain, a near-infrared fluorescent, non-specific cell staining that allows for the calculation of cell number in each well. Permeabilization of cells is necessary for DNA staining, but in the “classic” version of C5b-9 assay, cells are never permeabilized. To test whether the permeabilization step could affect C5b-9 staining, two detections of C5b-9 fluorescence were performed: one after the secondary antibody staining (800 nm), and the other after permeabilization and DNA staining (both 700 and 800 nm). No differences in C5b-9 signal were observed under these two conditions.

Each sample and each condition were tested in triplicate as follows:
Plate 1: resting cells; medium, control serum, aHUS1 acute, aHUS1 acute+sCR1, aHUS 2 acute, secondary antibody 1/600, left: in house blocking buffer, right: Odyssey commercial blocking buffer (FIG. 5A).
Plate 2: ADP-activated cells; medium, control serum, aHUS1 acute, aHUS1 acute+sCR1, aHUS 2 acute, secondary antibody 1/600, left: in house blocking buffer, right: Odyssey commercial blocking buffer (FIG. 5B).
Plate 3: resting cells; medium, control serum, aHUS1 acute, aHUS1 acute+sCR1, aHUS 2 acute, secondary antibody 1/1200, left: in house blocking buffer, right: Odyssey commercial blocking buffer (FIG. 5C).
Plate 4: ADP-activated cells; medium, control serum, aHUS1 acute, aHUS1 acute+sCR1, aHUS2 acute; secondary antibody 1/1200, left: in house blocking buffer, right: Odyssey commercial blocking buffer (FIG. 5D).

Results summarized in Table 7 show that serum from aHUS patients induced more C5b-9 deposits on either resting or ADP-activated HMEC-1 cells than control serum, and deposits were greatly reduced by sCR1. The best results were obtained with a 1:1200 dilution of the secondary antibody and were very comparable with that obtained with the “classic” assay using another aliquot of serum from the same patients. The only exception was that patient aHUS 2 showed a lower increase of C5b-9 deposits (percentage of C5b-9 deposits with control serum) on ADP-activated HMEC-1 cells with the new assay than with the “classic” assay (a previously thawed serum aliquot was used for this patient).

TABLE 7
C5b-9 deposits expressed as a percentage of deposits induced by the
corresponding control serum run in parallel.

| | resting cells | | | ADP-activated cells | | |
|---|---|---|---|---|---|---|
| | secondary | secondary | classic | secondary | secondary | classic |
| patient | Ab 1:600 | Ab 1:1200 | method | Ab 1:600 | Ab 1:1200 | method |
| aHUS 1 acute phase | 401 | 276 | 350 | 215 | 239 | 293 |
| aHUS 1 acute phase + | 162 | 122 | | 73 | 89 | |
| sCR1 150 μg/ml | | | | | | |
| aHUS 2 acute phase | 659 | 335 | 309 | 170 | 133 | 361 |

### Example 4: Evaluation of Patient-Derived Samples with the Automated C5b-9 Deposition Assay and HMEC-1 Short Term (16 and 24 Hours) Culture

This Example describes the use of the automated C5b-9 deposition assay with serum samples from aHUS patients (#3: aHUS patient on eculizumab treatment; #4: aHUS patient under remission and not being treated; #5: aHUS patient on eculizumab treatment) and healthy controls (serum pooled from 20 healthy subjects).

HMEC-1 cells were seeded at 15,000 per well in 96-well microplates and used either after overnight (about 16 hours) or 24 hours of culture. HMEC-1 monolayers were washed three times with test medium (HBSS: 137 mmol/l NaCl, 5.4 mmol/l KCl, 0.7 mmol/l Na₂HPO₄, 0.73 mmol/l KH₂PO₄, 1.9 mmol/l CaCl₂, 0.8 mmol/1l MgSO₄, 28 mmol/l Trizma base pH 7.3, 0.1% dextrose; with 0.5% BSA) and then activated with 10 μM ADP (Sigma-Aldrich) in test medium for 10 minutes (ADP-activated cells) or incubated with test medium alone (resting cells). Following this, cells were washed three times with test medium and then incubated for 4 hours with serum from control or from 3 different patients with aHUS diluted 1:2 with test medium (final volume: 75 μL). At the end of the incubation step HMEC-1 were washed twice with PBS, fixed in 3% paraformaldehyde, then washed again twice with PBS. Cells were blocked for one hour with 100 μL of commercial blocking buffer (Odyssey blocking buffer, LI-COR), followed by staining with rabbit anti-human complement C5b-9 complex (Calbiochem) followed by the secondary antibody IRDye 800 CW goat anti-rabbit IgG (H+L) (LI-COR), at 1:800 dilution.

To normalize the fluorescence intensity by cell number, after the acquisition of secondary antibody staining, cells were permeabilized with PBS 1×+0.1% Triton X-100 and then challenged with CellTag 700 Stain, a near-infrared fluorescent, non-specific cell staining that allowed calculation of the cell number in each well. Thereafter, detection of C5b-9 fluorescence was done at 800 nm, and the detection of CellTag 700 Stain at 700 nm. The signal at 800 nm was corrected for the signal at 700 nm (cell number). The corrected signal from wells with HMEC-1 incubated with the control pool serum was taken as 100% and results were expressed as % of the control. Each sample and each condition was tested in triplicate and the mean of the three replicates was calculated.

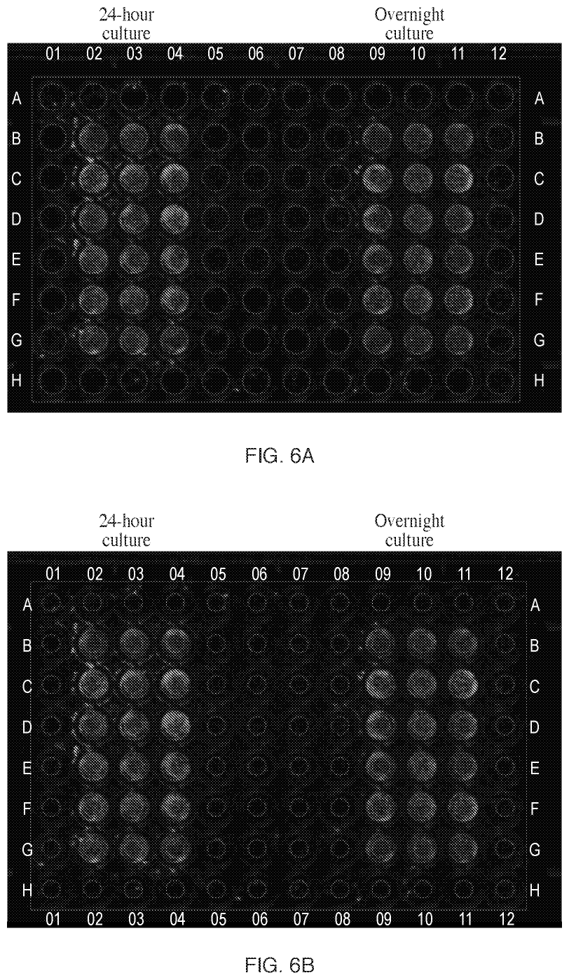

The intensity of the signal was first detected using a grid corresponding to the whole area of each well (standard grid, centered on CellTag 700 Stain at 700 nm that labels HMEC-1 cell nuclei and cytoplasm, FIG. 6A). However, the red fluorescence distribution of the CellTag 700 Stain in each well was more homogeneous in the central areas of the wells than in the peripheral areas. Thus, the analysis was repeated by using another grid that analyzed only the central area of the well (reduced grid, FIG. 6B).

There was good concordance among the results obtained with the automated test and HMEC-1 cultured overnight and previous results obtained using the same serum samples with the “classic” C5b-9 deposition test (96-hour HMEC-1 culture and confocal microscopy detection of C5b-9 deposits) (Table 8). Indeed with both methods, patients #3 and #5 (on eculizumab treatment) showed low C5b-9 deposits and patient #4 (in remission; no treatment) showed higher than normal C5b-9 deposits on resting and ADP-activated HMEC-1 cells. Notably, patient #4 was in apparent clinical remission, however the test was positive for C5b-9 deposits on resting HMEC-1 cells both with the classic and automated method. These results indicate that the serum-induced C5b-9 deposition test may highlight subclinical disease activity, which will be of great relevance for chronic monitoring of aHUS patients.

TABLE 8
C5b-9 deposits expressed as percentage of deposits induced by the
corresponding control serum run in parallel. Overnight plating -
15,000 cells/well.

| | resting cells | | | activated cells | | |
|---|---|---|---|---|---|---|
| | | auto- | | | auto- | |
| | | mated | method | | mated | method |
| | classic | standard | reduced | classic | standard | reduced |
| patient | method | grid | grid | method | grid | grid |
| aHUS #3 | 83% | 111% | 121% | 83% | 115% | 120% |
| (eculizumab) | | | | | | |
| aHUS #4 | 253% | 468% | 718% | 260% | 491% | 825% |
| (untreated) | | | | | | |
| aHUS #5 | 38% | 91% | 93% | 35% | 108% | 127% |
| (eculizumab) | | | | | | |

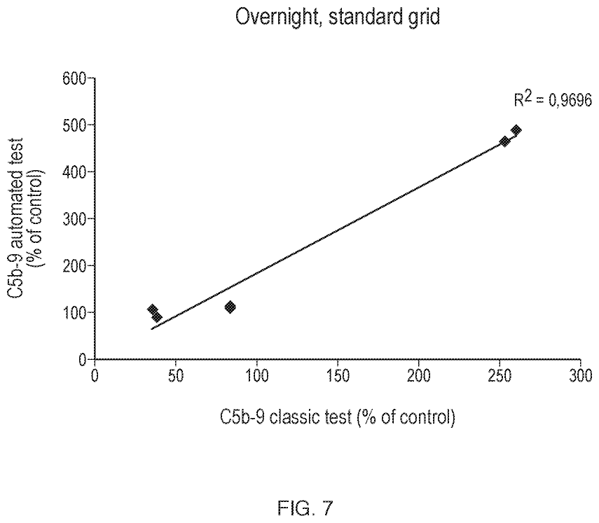

The use of the “standard” vs. the “reduced size” grid in the analysis did not substantially affect the results. However a better correlation between results of the “classic” and the “automated” tests was observed with the standard grid (R²: 0.97, FIG. 7) than with the reduced grid (R²: 0.95).

A similar experiment was conducted with HMEC-1 cells cultured for 24 hours (Table 9), and results were similar to those obtained with cells cultured overnight. Good concordance was observed among results obtained with the automated test and previous results obtained using the same serum samples with the “classic” C5b-9 deposition test.

TABLE 9
C5b-9 deposits expressed as percentage of deposits induced by the
corresponding control serum run in parallel. 24 hours plating -
15,000 cells/well.

| | resting cells | | | activated cells | | |
|---|---|---|---|---|---|---|
| | | auto- | | | auto- | |
| | | mated | method | | mated | method |
| | classic | standard | reduced | classic | standard | reduced |
| patient | method | grid | grid | method | grid | grid |
| aHUS #3 | 83% | 108% | 116% | 83% | 114% | 121% |
| (eculizumab) | | | | | | |
| aHUS #4 | 253% | 330% | 489% | 260% | 405% | 594% |
| (untreated) | | | | | | |
| aHUS #5 | 38% | 90% | 89% | 35% | 84% | 89% |
| (eculizumab) | | | | | | |

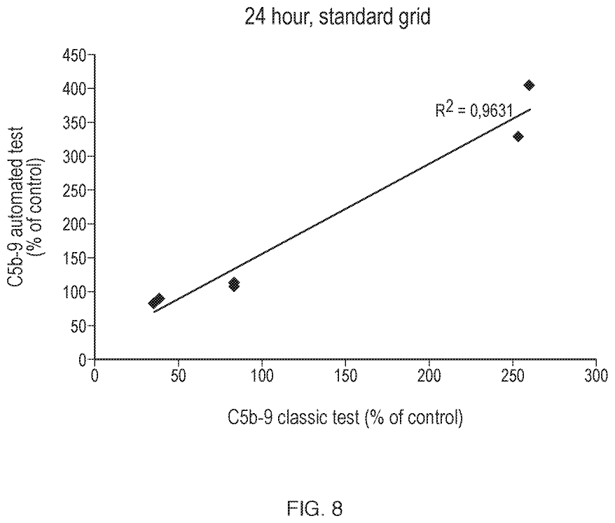

The use of the “standard” vs the “reduced size” grid in the analysis did not substantially affect the results (correlation among classic and automated test: standard grid R²: 0.96, FIG. 8; reduced grid R²: 0.96).

Based on the above results, subsequent experiments used the standard grid covering the whole area of each well for the analysis of the fluorescence signal.

### Example 5: Validation of Automated C5b-9 Deposition Assay

In this example, the automated C5b-9 deposition assay was validated using serum samples from three additional aHUS patients.

HMEC-1 cells were seeded at 15,000 per well in 96-well microplates and used after overnight (about 16 hours) or 24 hour culture. HMEC-1 cells (either resting or ADP-activated) were incubated with sera from 3 additional aHUS patients: #6 with aHUS, acute phase before any treatment; #7 with aHUS, remission, no treatment; and #8 with aHUS on eculizumab treatment. A pool of sera from 20 healthy subjects was studied in parallel as control. Samples were run in triplicate and analyzed as described above using the standard grid.

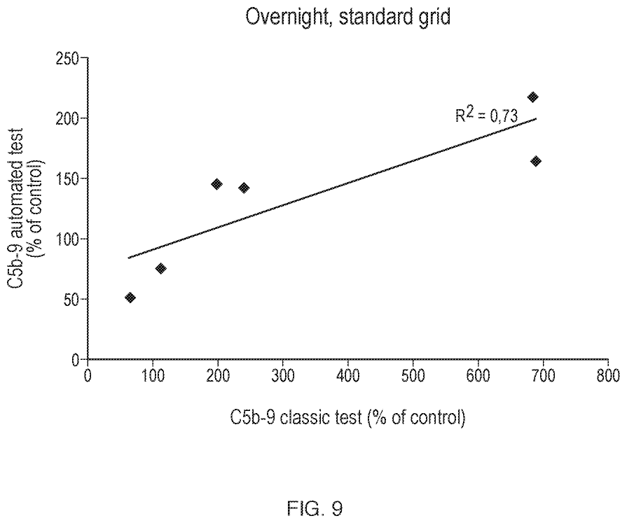

The results obtained with serum from these additional 3 patients confirmed a good concordance between the results of the classic and the automated tests performed on HMEC-1 cultured overnight (Table 10 and FIG. 9). Of note, patients #6 and #7 showed increased C5b-9 deposits on resting and ADP-activated HMEC-1 with both tests.

TABLE 10
C5b-9 deposits expressed as percentage of deposits
induced by the corresponding control serum run in
parallel. Overnight plating - 15,000 cells/well.

| | resting cells | | activated cells | |
|---|---|---|---|---|
| | automated | method | automated | method |
| | classic | standard | classic | standard |
| patient | method | grid | method | grid |
| aHUS #6 (untreated) | 688% | 165% | 684% | 218% |
| aHUS #7 (untreated) | 199% | 146% | 240% | 143% |
| aHUS #8 (eculizumab) | 65% | 52% | 112% | 76% |

### EQUIVALENTS

The skilled artisan will recognize, or be able to ascertain using no more than routine experimentation, many equivalents of the specific embodiments disclosed herein. Such equivalents are intended to be encompassed by the following claims.

SEQUENCE SUMMARY

amino acid sequence of heavy chain CDR1 of
eculizumab (as defined under combined
Kabat-Chothia definition)
SEQ ID NO: 1
GYIFSNYWIQ
amino acid sequence of heavy chain CDR2 of
eculizumab (as defined under Kabat definition)
SEQ ID NO: 2
EILPGSGSTEYTENFKD
amino acid sequence of the heavy chain CDR3 of
eculizumab (as defined under combined Kabat
definition).
SEQ ID NO: 3
YFFGSSPNWYFDV
amino acid sequence of the light chain CDR1 of
eculizumab (as defined under Kabat definition)
SEQ ID NO:4
GASENIYGALN
amino acid sequence of light chain CDR2 of
eculizumab (as defined under Kabat definition)
SEQ ID NO: 5
GATNLAD
amino acid sequence of light chain CDR3 of
eculizumab (as defined under Kabat definition)
SEQ ID NO: 6
QNVLNTPLT
amino acid sequence of heavy chain variable
region of eculizumab
SEQ ID NO: 7
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSS
amino acid sequence of light chain variable
region of eculizumab, BNJ441 antibody, and
BNJ421 antibody
SEQ ID NO: 8
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK
amino acid sequence of heavy chain constant
region of eculizumab and BNJ421 antibody
SEQ ID NO: 9
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLGK
amino acid sequence of entire heavy chain of
eculizumab
SEQ ID NO: 10
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
amino acid sequence of entire light chain of
eculizumab, BNJ441 antibody, and BNJ421 antibody
SEQ ID NO: 11
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
amino acid sequence of heavy chain variable
region of BNJ441 antibody and BNJ421 antibody
SEQ ID NO: 12
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS
amino acid sequence of heavy chain constant
region of BNJ441 antibody
SEQ ID NO: 13
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK
amino acid sequence of entire heavy chain of
BNJ441 antibody
SEQ ID NO: 14
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSV [symbol] HEALH [symbol] HYTQKSLSLS
LGK
amino acid sequence of IgG2 heavy chain constant
region variant comprising YTE substitutions
SEQ ID NO: 15
ASTKGPSVFPLAPCSRSTSESTAALGCLVKDYFPEPVTVSWNSGALTSGV
HTFPAVLQSSGLYSLSSVVTVTSSNFGTQTYTCNVDHKPSNTKVDKTVER
KCCVECPPCPAPPVAGPSVFLFPPKPKDTLYITREPEVTCVVVDVSHEDP
EVQFNWYVDGMEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK
amino acid sequence of entire heavy chain of
eculizumab variant comprising heavy chain constant
region depicted in SEQ ID NO: 15 (above)
SEQ ID NO: 16
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVTSSNFGT
QTYTCNVDHKPSNTKVDKTVERKCCVECPPCPAPPVAGPSVFLFPPKPKD
TLYITREPEVTCVVVDVSHEDPEVQFNWYVDGMEVHNAKTKPREEQFNST
FRVVSVLTVVHQDWLNGKEYKCKVSNKGLPAPIEKTISKTKGQPREPQVY
TLPPSREEMTKNQVSLTCLVKGFYPSDIAVEWESNGQPENNYKTTPPMLD
SDGSFFLYSKLTVDKSRWQQGNVFSCSVMHEALHNHYTQKSLSLSPGK
amino acid sequence of light chain CDR1 of
eculizumab (as defined under Kabat definition)
with glycine to histidine substitution at
position 8 relative to SEQ ID NO: 4
SEQ ID NO: 17
GASENIYHALN
depicts amino acid sequence of heavy chain CDR2
of eculizumab in which serine at position 8
relative to SEQ ID NO: 2 is substituted with
histidine
SEQ ID NO: 18
EILPGSGHTEYTENFKD
amino acid sequence of heavy chain CDR1 of
eculizumab in which tyrosine at position 2
(relative to SEQ ID NO: 1) is substituted
with histidine
SEQ ID NO: 19
GHIFSNYWIQ
amino acid sequence of entire heavy chain of
BNJ421 antibody
SEQ ID NO: 20
QVQLVQSGAEVKKPGASVKVSCKASGHTSNYWIQWVRQAPGQGLEWMGEI
LPGSGHTEYTENFKDRVTMTRDTSTSTVYMELSSLRSEDTAVYYCARYFF
GSSPNWYFDVWGQGTLVTVSSASTKGPSVFPLAPCSRSTSESTAALGCLV
KDYFPEPVTVSWNSGALTSGVHTFPAVLQSSGLYSLSSVVTVPSSNFGTQ
TYTCNVDHKPSNTKVDKTVERKCCVECPPCPAPPVAGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSV [symbol] HEALH [symbol] HYTQKSLSLSL
GK
amino acid sequence of full light chain of BNJ383
antibody without signal peptide
SEQ ID NO: 21
DIQMTQSPSSLSASVGDRVTITCRASESVDSYGNSFMHWYQQKPGKAPKL
LYIRASNLESGVPSRFSGSGSGTDFTLTISSLQPEDFATYYCQQSNEDPY
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
amino acid sequence of light chain variable
region of BNJ383 antibody
SEQ ID NO: 22
DIQMTQSPSSLSASVGDRVTITCRASESVDSYGNSFMHWYQQKPGKAPKL
LIYRASNLESGVPSRFSGSGSGTDFTLTISSLQPEDFATYYCQQSNEDPY
TFGGGTKVEIKR
amino acid sequence of light chain variable region
sequence Kabat LCDR1 of BNJ383 antibody
SEQ ID NO: 23
RASESVDSYGNSFMH
amino acid sequence of light chain variable region
sequence Kabat LCDR2 of BNJ383 antibody
SEQ ID NO: 24
RASNLES
amino acid sequence of light chain variable region
sequence Kabat LCDR3 of BNJ383 antibody
SEQ ID NO: 25
QQSNEDPYT
amino acid sequence of full heavy chain of BNJ383
antibody without signal peptide
SEQ ID NO: 26
QVQLVQSGAEVKKPGASVKVSCKASGYTFTDYSMDWVRQAPGQGLEWMGA
IHLNTGYTNYNQKFKGRVTMTRDTSTSTVYMELSSLRSEDTAVYYCARGF
YDGYSPMDYWGQGTTVTVSSASTKGPSVFPLAPCSRSTSESTAALGCLVK
DYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK
amino acid sequence of heavy chain variable region
of BNJ383 antibody
SEQ ID NO: 27
QVQLVQSGAEVKKPGASVKVSCKASGYTFTDYSMDWVRQAPGQGLEWMGA
IHLNTGYTNYNQKFKGRVTMTRDTSTSTVYMELSSLRSEDTAVYYCARGF
YDGYSPMDYWGQGTTVTVSS
amino acid sequence of heavy chain variable region
sequence Kabat LCDR1 of BNJ383 antibody
SEQ ID NO: 28
DYSMD
amino acid sequence of heavy chain variable region
sequence Kabat LCDR2 of BNJ383 antibody
SEQ ID NO: 29
AIHLNTGYTNYNQKFKG
amino acid sequence of heavy chain variable region
sequence Kabat LCDR3 of BNJ383 antibody
SEQ ID NO: 30
GFYDGYSPMDY

We claim:

1. A method for measuring complement C5b-9 deposition comprising:
(a) contacting ex vivo a biological sample obtained from a patient who has or is suspected of having a complement-associated disorder with disease-relevant cells;
(b) assessing levels of C5b-9 deposition on the cells; and
(c) normalizing levels of C5b-9 deposition by cell number.

2. A method for determining whether a patient with a complement-associated disorder would benefit from treatment with an inhibitor of C5, the method comprising:
(a) incubating a biological sample obtained from the patient with and without an inhibitor of C5;
(b) contacting ex vivo endothelial cells with the biological sample from step (a);
(c) assessing levels of C5b-9 deposition on the cells; and
(d) normalizing levels of C5b-9 deposition by cell number,
wherein less C5b-9 deposition with the biological sample incubated with the inhibitor compared to without the inhibitor indicates the patient is likely to benefit from treatment with the inhibitor.

3. A method for determining whether a patient with a complement-associated disorder is likely to benefit from treatment with eculizumab, the method comprising:
(a) incubating a biological sample obtained from the patient with and without eculizumab;
(b) contacting ex vivo endothelial cells with the biological sample from step (a);
(c) assessing levels of C5b-9 deposition on the cells; and
(d) normalizing levels of C5b-9 deposition by cell number,
wherein less C5b-9 deposition with the biological sample incubated with eculizumab compared to without eculizumab indicates the patient is likely to benefit from treatment with eculizumab.

4. A method for determining whether a patient with atypical hemolytic uremic syndrome (aHUS) is likely to benefit from treatment with eculizumab, the method comprising:
(a) incubating a biological sample obtained from the patient with and without eculizumab;
(b) contacting ex vivo endothelial cells with the biological sample from step (a);
(c) assessing levels of C5b-9 deposition on the cells; and
(d) normalizing levels of C5b-9 deposition by cell number,
wherein less C5b-9 deposition with the biological sample incubated with eculizumab compared to without eculizumab indicates the patient is likely to benefit from treatment with eculizumab.

5. A method for monitoring a patient who has a complement-associated disorder and is being treated with an inhibitor of C5, the method comprising:
(a) contacting ex vivo endothelial cells with a biological sample from the patient and a control sample;
(b) assessing levels of C5b-9 deposition on the cells;
(c) normalizing levels of C5b-9 deposition by cell number; and
(d) increasing the dose of the inhibitor administered to the patient if C5b-9 deposition with the biological sample from the patient being treated with the inhibitor is greater compared to C5b-9 deposition with the control sample.

6. The method of claim 5, wherein if the patient is administered an increased dose of the inhibitor, steps (a)-(c) are repeated to determine whether the increased dose is sufficient to normalize levels of C5b-9 deposition on the cells.

7. A method of treating a complement-associated disorder in a patient determined to be responsive to an inhibitor of C5 or eculizumab according to the method of any one of claims 1-4, the method comprising administering to the patient a therapeutically-effective amount of the inhibitor or eculizumab.

8. The method of claim 2, 5, or 6, wherein the inhibitor of C5 is an antibody, such as eculizumab.

9. The method of any one of the preceding claims, wherein the cells are cultured on a solid platform, such as a microplate.

10. The method of claim 9, wherein the solid platform is a 96-well microplate.

11. The method of any one of the preceding claims, wherein the disease-relevant cells are selected from the group consisting of endothelial cells, retinal pigment epithelial cells, chondrocytes, neurons, glial cells, skeletal muscle cells, and cardiomyocytes.

12. The method of claim 11, wherein the disease-relevant cells are endothelial cells selected from the group consisting of human microvascular endothelial cells from dermal origin, human umbilical vein endothelial cells, endothelial cells from foreskin, and endothelial cells from liver adenocarcinoma.

13. The method of any one of the preceding claims, wherein the cells are plated at a density of about 5,000 to about 6,000 cells per well and cultured until confluent.

14. The method of any one of the preceding claims, wherein the cells are plated at a density of about 10,000 cells to about 12,500 cells per well and cultured until confluent.

15. The method of any one of claims 1-12, wherein the cells are plated at a density of about 15,000 cells per well cultured until confluent.

16. The method of any one of the preceding claims, wherein cells are confluent before being contacted with the biological sample.

17. The method of any one of the preceding claims, wherein the biological sample is serum.

18. The method of claim 17, wherein the serum is from a patient with aHUS, a patient in remission, or an eculizumab-naïve patient.

19. The method of any one of the preceding claims, wherein the cells are activated with adenosine 5′-diphosphate, thrombin, or lipopolysaccharide.

20. The method of any one of the preceding claims, wherein the cells are contacted with the biological sample for about 1.5 hours to about 4 hours.

21. The method of any one of the preceding claims, wherein the cells are incubated with a fixative such as paraformaldehyde after the contacting step but before the assessing step.

22. The method of any one of the preceding claims, wherein the levels of C5b-9 deposition are assessed using an anti-C5b-9 antibody.

23. The method of claim 20, wherein the anti-C5b-9 antibody is detected with a secondary antibody comprising a detectable label such as a dye.

24. The method of any one of the preceding claims, wherein the levels of C5b-9 deposition are assessed using an On-cell Western assay.

25. The method of claim 23, wherein the cells are permeabilized after the anti-C5b-9 antibody is detected with the secondary antibody.

26. The method of claim 23, wherein the cells are permeabilized before the anti-C5b-9 antibody is detected with the secondary antibody.

27. The method of claim 25 or 26, wherein, following permeabilization, the cells are incubated with an agent that accumulates in the nucleus, such as an agent that stains DNA.

28. The method of claim 27, wherein the agent is selected from the group consisting of:
CellTag 700 Stain, DAPI, acridine orange, Hoechst 33342 Dye, Hoechst 33258, SYTOX Green nucleic acid stain, and Vybrant DyeCycle stain.

29. The method of any one of the preceding claims, wherein one or more steps are automated.

30. The method of any one of claims 1-3, 5-17, and 19-29, wherein the patient has atypical hemolytic uremic syndrome, STEC-HUS, diabetes, lupus nephritis, vasculitis, or chronic allograft rejection.
